(19) United States
(12) Patent Application Publication
(10) Pub. No.: US 2023/0233321 A1
(43) Pub. Date: Jul. 27, 2023

(54) DEVICES AND METHODS FOR CERCLAGE OF LUMENAL SYSTEMS

(71) Applicants: The United States of America, As Represented by the Secretary, Department of Health and Human Servic, Bethesda, MD (US); Transmural Systems LLC, Andover, MA (US)

(72) Inventors: Robert J. Lederman, Bethesda, MD (US); Christopher G. Bruce, Bethesda, MD (US); Morgan House, Andover, MA (US); Nasser Rafiee, Andover, MA (US)

(21) Appl. No.: 18/151,601

(22) Filed: Jan. 9, 2023

Related U.S. Application Data
(63) Continuation of application No. PCT/US2021/041310, filed on Jul. 12, 2021.
(60) Provisional application No. 63/050,270, filed on Jul. 10, 2020.

(51) Int. Cl. A61F 2/24; A61N 1/05; A61B 5/0215

(52) U.S. Cl. CPC A61F 2/2442; A61B 5/0215; A61N 1/0563; A61F 2210/0066

(57) ABSTRACT

The present disclosure provides embodiments of devices that are useful in the structural remodeling of various parts of the cardiovascular system, most notably the heart. Certain of the disclosed devices relate to RAMIN procedures (“remodeling and ablation using myocardial interstitial navigation”). RAMIN procedures, as described herein, represent a new family of non-surgical catheter-based procedures in order to accomplish ablation, drug delivery, re-shaping, pacing, and related structural heart interventional procedures, as desired.

### CROSS-REFERENCE TO RELATED APPLICATION

This patent application is a continuation of and claims the benefit of priority to International Application No. PCT/US2021/041310 filed Jul. 12, 2021, which in turn claims the benefit of priority to U.S. Provisional Pat. Application No. 63/050,270, filed Jul. 10, 2020. Each of the aforementioned patent applications is hereby incorporated by reference in its entirety for all purposes.

### FIELD

This disclosure relates generally to interventional devices for changing the shape of portions of lumenal systems.

### BACKGROUND

Many devices and systems of the prior art for cardiac remodeling. The present disclosure provides solutions for these and other problems.

### SUMMARY OF THE DISCLOSURE

Advantages of the present disclosure will be set forth in and become apparent from the description that follows. Additional advantages of the disclosure will be realized and attained by the methods and systems particularly pointed out in the written description and claims hereof, as well as from the appended drawings.

In some implementations, the disclosure provides an implant configured to traverse a passageway defined through tissue about a cardiac chamber of a heart. The implant includes an elongate flexible tether having two ends formed into a loop, and a lock body disposed over the two ends of the tether. The lock body can be configurable to releasably engaging the elongate flexible tether. The implant can further include first and second tubular limbs extending outwardly from the lock over the elongate flexible tether along the loop toward each other.

In some implementations, the implant can be structurally compliant or flexible and can change in length in response to movements of the heart. This can be accomplished in a variety of manners, such as by including one or more segments of the implant that elongate and shrink in transverse dimension when placed under increasing tension. The material of the implant can be selected to do this. In some implementations, one or more segments of the implant can be formed from a compliant material in the shape of a leaf spring or a tension spring, or have a helical shape that that unwinds slightly and elongates when placed under tension. In another implementation, an implant body can be provided with an elongate tether that is stitched across the implant body, and along the body, such that the elongate tether forms a sinusoidal, sawtooth, or square wave shape, for example. When the elongate tether is tensioned, one or more segments of the implant body can contract axially and expand radially.

If desired, the first and second tubular limbs can be of different diameters. The first and second tubular limbs can have tapered distal ends. A distal end of the first tubular limb can slide within a distal end of the second tubular limb along the loop of the elongate flexible tether such that the first and second tubular limbs overlap. At least one of the first and second tubular limbs can include a plurality of radiopaque markers along its length. The plurality of radiopaque markers can be disposed along the length of said at least one of the first and second tubular limbs in a predetermined pattern in order to facilitate measurement of the implant under visualization.

In some implementations, at least one of the first and second tubular limbs can include at least one pacing electrode to stimulate cardiac tissue. The implant can further include a controller coupled to the at least one pacing electrode to provide at least one of pacing, defibrillation, measurement and control. If desired, the elongate flexible tether can include an antenna, such as a loop antenna that conducts signals to and from the controller. If desired, the implant can further include a controller and a reservoir containing a beneficial agent, wherein the controller can be coupled to a dispenser that is coupled to the reservoir to dispense the beneficial agent. The beneficial agent can include a medication. The beneficial agent can include a gene therapy material. The beneficial agent can include living cells to seed at least one location of the heart that is damaged. At least one of the first and second tubular limbs can include at least one sensor to sense at least one biological parameter. The at least one sensor can include at least one pressure sensor to sense blood pressure. The at least one sensor can include at least one of: a chemical sensor, a distance sensor, a sensor having circuitry to detect electro physiological data, a movement sensor, and a location sensor. The elongate flexible tether can include radiopaque material along its length. The elongate flexible tether can be a hollow braided suture, and the radiopaque material within the elongate flexible tether can include a radiopaque wire disposed within a length of heat shrunk polymeric tube that resides within a hollow core of the elongate inner tether. The implant lock can define at least one distal opening therein. The at least one distal opening can be connected to the first and second tubular limbs.

In further accordance with the disclosure, an implant is provided that includes an elongate inner tether having a proximal end and a distal end. The proximal end of the elongate inner tether can terminate in a loop. The implant can include an outer tubular body surrounding the elongate inner tether along at least a portion of the length of the inner tether. The outer tubular body can be shorter in length than the elongate inner tether. In some implementations, the outer tubular body can be configured to shorten in length and increase in transverse dimension when it is compressed along an axial direction. If desired, the outer tubular body can include a braided structure. In some implementations, the elongate inner tether can be threaded intermittently through the outer tubular body. In some implementations, the outer tubular body can include a resilient member. If desired, the outer tubular body can include a shape memory material, a resilient member, and/or a coil spring. In some implementations, the outer tubular body can include a plurality of radiopaque markers along its length. The plurality of radiopaque markers disposed along the length of the outer tubular body can be arranged at predetermined intervals to facilitate measurement of the implant under visualization.

In some implementations, the outer tubular body can include at least one pacing electrode to stimulate cardiac tissue. If desired, the implant can further include a controller coupled to the at least one pacing electrode to provide at least one of pacing, defibrillation, measurement and control. The implant can include an antenna, such as a loop antenna or dipole antenna that conducts signals to and from the controller. If desired, the implant can include a controller and a reservoir containing a beneficial agent. The controller can be coupled to a dispenser that is coupled to the reservoir to dispense the beneficial agent. If desired, the beneficial agent can include one or more of a medication, a gene therapy material, and living cells to seed at least one location of the heart that is damaged.

In some implementations, the outer tubular body can include at least one sensor to sense at least one biological parameter. At least one sensor can include at least one pressure sensor to sense blood pressure. The at least one sensor can include at least one of: a chemical sensor, a distance sensor, a sensor having circuitry to detect electro physiological data, a movement sensor, and a location sensor.

In some embodiments, the elongate inner tether can include radiopaque material along its length. The elongate inner tether can be a hollow braided suture, and the radiopaque material within the elongate inner tether can include a radiopaque wire disposed within a length of heat shrunk polymeric tube that resides within a hollow core of the elongate inner tether. The implant can further include an implant lock configured to lock the implant into a loop form.

In some embodiments, the disclosure provides a method of reducing the dimensional size of a portion of a patient’s heart. The method can include advancing a guidewire into a patient’s circulatory system and into the patient’s heart, advancing the guidewire through the myocardium to define a passageway around at least a portion of the heart between an outer surface of the heart and an inner surface of the heart, exchanging the guidewire with an implant including a tensioning element so that the tensioning element traverses the passageway, advancing a lock over the tensioning element, applying tension to the tensioning element to change the dimensional size of a portion of the heart, and locking the lock to maintain the tension in the tensioning element.

In some implementations, the method can further include unlocking the lock, adjusting the tension in the tensioning element, and relocking the lock. In some implementations, the tensioning element can be of a larger diameter than the guidewire. If desired, the lock can include two elongate tubular limbs coupled to a lock body, and the method can further include advancing the two elongate tubular limbs along the tensioning element in order to increase the effective diameter of the tensioning element. The distal ends of the two elongate tubular limbs can be configured to slide past one another when traversing the tensioning element and overlap. The distal end of a first of the two elongate tubular limbs can be configured to be received inside the distal end of the second of the two elongate tubular limbs.

In some implementations, the lock can include an electrode array coupled to a signal generator configured to effectuate cardiac pacing, and the method can further include performing a cardiac pacing function using the electrode array and the signal generator. The pacing function can effectuate depolarization of the myocardium. In some implementations, the pacing function can include synchronously depolarizing the basal left ventricle. If desired, the pacing function can include effectuating a pacing function on the patient’s HIS bundle.

In some implementations, the method can further include delivering a beneficial agent to a target location in the patient’s myocardium. In some implementations, delivering a beneficial agent can include performing a chemoablation procedure to debulk the myocardium. In some implementations, the beneficial agent can include one or more of (i) a pharmaceutical composition, (ii) light, and (iii) ultrasonic energy, for example.

In some implementations, the elongate passageway through the myocardium traverses a portion of the septum. If desired, the method can further include delivering a beneficial agent as described elsewhere herein to a target location in the patient’s septum. If desired, the delivering the beneficial agent can include performing a chemoablation procedure to debulk the septum.

In other implementations, the method can include defining an elongate passageway that traverses a path around a portion of at least one of the patient’s ventricles. If desired, the elongate passageway can traverses a path around a portion of both of the patient’s ventricles. If desired, the elongate passageway can encircle one of the patient’s ventricles at the basal level. In other implementations, the elongate passageway can encircle one of the patient’s ventricles at the mid-myocardial level. If desired, the elongate passageway can encircle the patient’s left ventricle.

In some implementations, the method can further include directing a second tensioning element through the patient’s myocardium and tensioning the second tensioning element to effectuate a further dimensional change to the patient’s heart. For example, multiple independent elongate passageways can be defined, and an implant can be installed along each elongate passageway.

In some embodiments, the guidewire can include an electrical conductor that is coated with a dielectric coating. An exposed region of the electrical conductor near a distal portion of the guidewire can be exposed and not coated with the dielectric coating, and the elongate passageway can be formed at least in part by ablating tissue by applying electrical power to the electrical conductor. If desired, electrical power is applied in a monopolar operating mode. In some implementations, electrical power can be applied in a bipolar operating mode. A return path for electrical current can be defined by a second conductor disposed near the exposed region of the electrical conductor. The exposed region of the electrical conductor can be located at a distal tip of the guidewire. The exposed region of the electrical conductor can be located on a side of the guidewire near a distal tip of the guidewire. The exposed region of the electrical conductor can be located on a side of the guidewire at a distal tip of the guidewire. If desired, the distal end region of the guidewire can include a bent section that is directed off a central longitudinal axis of the guidewire. In some implementations, the guidewire or a support catheter supporting the guidewire at least partially defines a longitudinal channel along at least a portion of its length configured to direct a fluid out of a distal end of the longitudinal channel to facilitate tissue dissection, and the method can further include directing a pressurized fluid through the longitudinal channel to help define the elongate passageway.

In some implementations, the elongate passageway can be formed at least in part by expanding an inflatable balloon coupled to a catheter that is disposed within the myocardium. The balloon can be introduced to an opening made into the myocardium by the guidewire. The balloon can be inflated to create an enlarged entry port into the myocardium to permit the introduction into the myocardium of at least one supporting catheter. The balloon can be coupled to an inflation catheter that is at least partially slidably disposed over the guidewire.

In some implementations, a method is provide that includes a distal end of a guidewire using a snare catheter, wherein the snare catheter includes an inflatable member disposed inside the snare, and further wherein inflation of the inflatable member causes the snare to expand. This can be done to bluntly dissect surrounding tissue to make room for the snare. The balloon can be deflated after the dissection occurs, and the snare catheter can then capture the guidewire and collapse to trap the guidewire. For example, this guidewire capturing step can occur in the myocardium. It can similarly be accomplished outside of the myocardium. The elongate passageway can be formed at least in part by directing a pressurized fluid to a target location within the myocardium.

Advancing the guidewire into the myocardium can include advancing a centripetal accessor catheter over the guidewire that helps direct the guidewire into the myocardium. The centripetal accessor catheter can include a radiopaque marker near its distal end that indicates the relative rotational position of the centripetal accessor catheter.

In some implementations, advancing the guidewire through the myocardium can include defining the passageway by advancing the guidewire through the myocardial tissue, wherein myocardial tissue is ablated at least in part to define the passageway. For example, the myocardial tissue can be ablated by applying electrical energy through the guidewire to energize an electrically uninsulated exposed distal end surface of the guidewire. The method can further include advancing a first supporting catheter disposed about the guidewire distally along a portion of the passageway created during the ablating step to surround a distal portion of the guidewire and provide column strength to the guidewire. These steps can be repeated until a passageway through the myocardial tissue is formed and completed.

In some embodiments, a distal end portion of the guidewire can include at least one visually enhanced marker visible under a visualization mode. Related methods can include visualizing the guidewire and myocardium under the visualization mode during the procedure to help control advancement of the guidewire through the myocardial tissue. The method can further include advancing a second supporting catheter over the first supporting catheter to further dilate the passageway. In addition, the method can further include withdrawing the first supporting catheter over the guidewire, leaving the guidewire and the second supporting catheter in place. As a result, an annular space is formed around the guidewire within the second supporting catheter, and a second guidewire can be inserted through the second supporting catheter alongside the first guidewire. At this point, the method can include withdrawing the second supporting catheter over the first guidewire and the second guidewire. Next, the first supporting catheter can be advanced over the first guidewire, and the second supporting catheter can once again be advanced over the first supporting catheter.

In some implementations, the passageway that is formed defines a complete loop that intersects itself. The distal end of the first guidewire can then be advanced distally so as to reenter the passageway to complete a loop. After this, a snare catheter can be introduced over the second guidewire to a location near where the distal end of the first guidewire has re-entered the passageway. The snare catheter can then be actuated in order to capture the distal end of the first guidewire, and the first guidewire can be withdrawn out of the patient using the snare catheter so that the first guidewire defines a loop about the passageway. The method can further includes externalizing the proximal and distal ends of the first guidewire. A distal end of the tensioning element can then be coupled to the proximal end of the first guidewire. The tensioning element can then be advanced about the path defined by the first guidewire until the tensioning element is located in a position to permit the lock to be introduced over the tensioning element.

In further accordance with the disclosure, a method is provided of treating a patient’s vasculature, including advancing a guidewire into a patient’s circulatory system and into a wall structure of the patient’s vasculature, advancing the guidewire through the wall structure to define a passageway along the wall structure between an outer surface of the wall structure and an inner surface of the wall structure, exchanging the guidewire with a tensioning element so that the tensioning element traverses the passageway, advancing a lock over the tensioning element, applying tension to the tensioning element, and locking the lock on the tensioning element. In some implementations, locking the tension in place can include advancing a knot along the tensioning element. The tensioning element can include a suture. The knot can be driven over first and second ends of the tensioning element to form a tensioned loop. The advancing of the lock and the locking of the lock can include advancing a crimp over first and second ends of the tensioning element to form a tensioned loop and crimping the crimp in place.

Preferably, the procedures set out herein are percutaneous and the tensioning element can be introduced by way of the patient’s circulatory system. In some implementations, the procedure can includes advancing a guidewire percutaneously through a wall of a blood vessel in the heart and through the myocardium to define the elongate passageway. The procedure can include advancing a guidewire percutaneously through a wall of a blood vessel around the wall of the blood vessel to define the elongate passageway. The blood vessel can include the abdominal aorta, and the passageway can be defined through a healthy portion of the abdominal aorta located above an aneurysm, and further wherein the method can further include coupling the tensioning element to an implant disposed in the abdominal aorta to prevent the implant from migrating. The implant can be located in a manner so as to at least partially span, or fully span, a compromised region of the aorta, such as a region of the aorta that includes an aneurysm.

In some implements, an implant can be introduced that includes a single limb, or outer tubular member, as desired, that includes a tensioning element disposed therethrough. The method can further include axially shortening the outer tubular member by applying tension to the tensioning tether. A transverse dimension of the outer tubular member can be configured to expand when it is contracted axially so as to increase its effective surface area to in turn spread stresses over a larger area of the myocardium to prevent the implant from pulling or cutting through myocardial tissue after it is implanted. Such a procedure will typically begin with a dissection process as disclosed herein to define a passageway to receive the implant, such as by using one or more support catheters. Regardless, when exchanging the guidewire with the tensioning element, a support catheter is either present over the guidewire already or one is introduced over the guidewire. A distal end of the guidewire that has been captured, for example, using a snare catheter is externalized and coupled, such as by way of crimped connection or another connection to a distal end of the tensioning tether. The tensioning tether is then withdrawn through the support catheter to permit the distal end of the tensioning tether to be similarly externalized. In some embodiments, a distal end of a retention tether can be coupled to the distal end of the guidewire, and the retention tether can be withdrawn through the supporting catheter. A proximal end of the retention tether can be attached to a distal end of the outer tubular member. The method can further include pulling the outer tubular member along a path defined by the support catheter while the support catheter is withdrawn to place the outer tubular member at a desired location in anatomy. A distal end of the tensioning tether can be directed through a proximal loop formed in a proximal end of the tensioning tether. The outer tubular member can be disposed between the proximal loop on a proximal end of the outer tubular member and the point where the proximal loop and distal end of the tensioning tether intersect at a distal end of the tether.

The disclosure further provides a guidewire that includes an electrically conductive core member surrounded by an insulating jacket. The guidewire can define an electrically uninsulated exposed distal end surface disposed at a bent distal section that is directed off a central longitudinal axis of a proximal portion of the guidewire. The electrically uninsulated exposed distal end surface can be located at a distal tip of the guidewire and can be axisymmetric with respect to a longitudinal axis of the bent distal section of the guidewire.

The electrically uninsulated exposed distal end surface can be located at a distal tip of the guidewire and not be axisymmetric with respect to a longitudinal axis of the bent distal section of the guidewire. The electrically uninsulated exposed distal end surface can be located proximal with respect to a distal tip of the guidewire and not be axisymmetric with respect to a longitudinal axis of the bent distal section of the guidewire. The disclosure further provides a catheter that includes the aforementioned guidewire disposed inside of a tubular member. The tubular member can include an exposed conductor at a distal end thereof coupled to a conductor extending to a proximal end region of the tubular member.

An electrosurgical system is similarly provided that includes a power source operably coupled to the guidewire, wherein the system is configured to operate in a monopolar operating mode. An electrosurgical system is also provided that includes a power source operably coupled to the aforementioned catheter, wherein the system is configured to operate in a bipolar operating mode and complete an electrical circuit from a distal tip of the guidewire to the distal tip of the tubular member. The guidewire and related method can include detecting and processing electrical signals received from cardiac tissue. Similarly, these devices can be sued to record or monitor intracardiac electrograms to help guide navigation through tissue. The catheter of the system can be coupled to a tube, such as a hypotube, that is in turn coupled to a fluid source. A related method is provided that includes using a catheter as described to dissect tissue at least in part by directing fluid from the fluid source out of a distal end of the hypotube. Saline or contrast fluid can similarly be directed out of a distal end of the hypotube.

The disclosure further provides a catheter that includes an elongate tubular member coupled to an inflatable member near a distal end of the catheter and a reservoir of inflation fluid, and a collapsible snare surrounding the inflatable member, wherein inflation of the inflatable member with inflation fluid causes the collapsible snare to expand. The collapsible snare is a single loop snare. The collapsible snare can be a multiple loop snare. The collapsible snare can be configured to remain open after the inflatable member is deflated.

The disclosure further provides a tensioning element expandable from a first, smaller effective diameter, to a second, larger effective diameter. The tensioning element can include a plurality of longitudinal rails configured to be separated from each other to effectuate expansion to a larger effective diameter. The tensioning element can include a core member and at least one tubular member disposed around the core member to increase the effective diameter of the tensioning element. The tensioning element can further include a plurality of markers visible under at least one visualization modality a long a length of the tensioning element. The tensioning element can further include an “L”-shaped lock disposed over first and second ends of the tensioning element.

It is to be understood that the foregoing general description and the following detailed description are exemplary and are intended to provide further explanation of the disclosed embodiments. The accompanying drawings, which are incorporated in and constitute part of this specification, are included to illustrate and provide a further understanding of the disclosed methods and systems. Together with the description, the drawings serve to explain principles of the disclosure.

### BRIEF DESCRIPTION OF THE FIGURES

FIGS. 1-7B illustrate use of a first implant in accordance with the disclosure.

FIGS. 8-21 illustrate use of a second implant in accordance with the disclosure.

FIGS. 22A-24B present additional devices in accordance with the disclosure.

FIGS. 25A-44 present aspects of an illustrative method in accordance with the disclosure.

### DETAILED DESCRIPTION OF PREFERRED EMBODIMENTS

Reference will now be made in detail to the present preferred embodiments of the disclosure, examples of which are illustrated in the accompanying drawings. The methods and corresponding steps of the disclosed embodiments will be described in conjunction with the detailed description of the system.

The present disclosure provides embodiments of devices that are useful in the structural remodeling of various parts of the cardiovascular system, most notably the heart. But, it will be appreciated that other portions of the anatomy can similarly be remodeled using the disclosed techniques, such as portions of the aorta, other lumens, vessels, and organs.

Certain of the disclosed devices relate to RAMIN procedures (“remodeling and ablation using myocardial interstitial navigation”). RAMIN procedures, as described herein, represent a new family of non-surgical catheter-based procedures in order to accomplish ablation, drug delivery, re-shaping, pacing, and related structural heart interventional procedures, as desired.

In transcatheter mitral cerclage annuloplasty as described in U.S. Pat. No. 10,433,962 (incorporated by reference herein in its entirety), the guidewire navigates coronary vein branches to allow a guidewire loop to encircle the mitral annulus and left ventricular outflow tract, to be exchanged for a permanent implant to apply tension and alter myocardial and mitral valve function.

In implementations of MIRTH (Myocardial Intramural Restraint by endovenous interstitial teTHer) procedures, as set forth herein, the guidewire is navigated inside the left ventricle muscle to create a deep sub-epicardial loop that encircles the left ventricle in order to apply tension and restrain or remodel the heart when it is pathologically dilated. The encirclement can be at the basal level, the mid-myocardial level, or any other. Once the guidewire navigates this trajectory, it is exchanged for a tension element (e.g, implant with tensioning tether) and a device to adjust or retain that tension. Because of its subepicardial trajectory, there is no risk of coronary vascular entrapment, and reduced risk of high grade atrioventricular block upon application of tension. Moreover, a subepicardial tension element obviates the risk of pull-through of anchor-based annuloplasty or ventriculoplasty devices.

The passageway or trajectory created using the MIRTH procedure can be further exploited to enable MIRTH based pacing to achieve permanent cardiac pacing or cardiac resynchronization therapy when the native cardiac conduction system fails at the level of the AV node, the bundle of His, or any other level requiring permanent pacing (wherein solitary right ventricular pacing causes or exacerbates cardiomyopathy). In implementations of SCIMITAR procedures (Suture via Coronary sinus with Interstitial myocardial navigation for MItral and Tricuspid Annular Reduction) a passageway is created that can encircle both ventricles.

In CEVICHE procedures (Catheter Endo-Venous myocardial Interstitial CHEmoablation), as described herein, the intraseptal cerclage trajectory is navigated to deliver a catheter for ablative drug delivery (“chemoablation”) such as ethanol or glacial acetic acid, for example to debulk the septum in patients at risk of left ventricular outflow tract obstruction complicating transcatheter mitral valve implantation, or in patients with hypertrophic cardiomyopathy. The CEVICHE procedure can also be used to ablate other pathological targets including the critical reentrant isthmus of ventricular tachycardia, or subvalvular tissue or membranes causing subvalvular pulmonary valve stenosis or subvalvular aortic valve stenosis. The procedures set forth herein distribute load relatively uniformly around the myocardium to minimize so-called “cheesecutting” or erosion.

In some implementations, the disclosure provides an implant configured to traverse a passageway defined through tissue about a cardiac chamber of a heart.

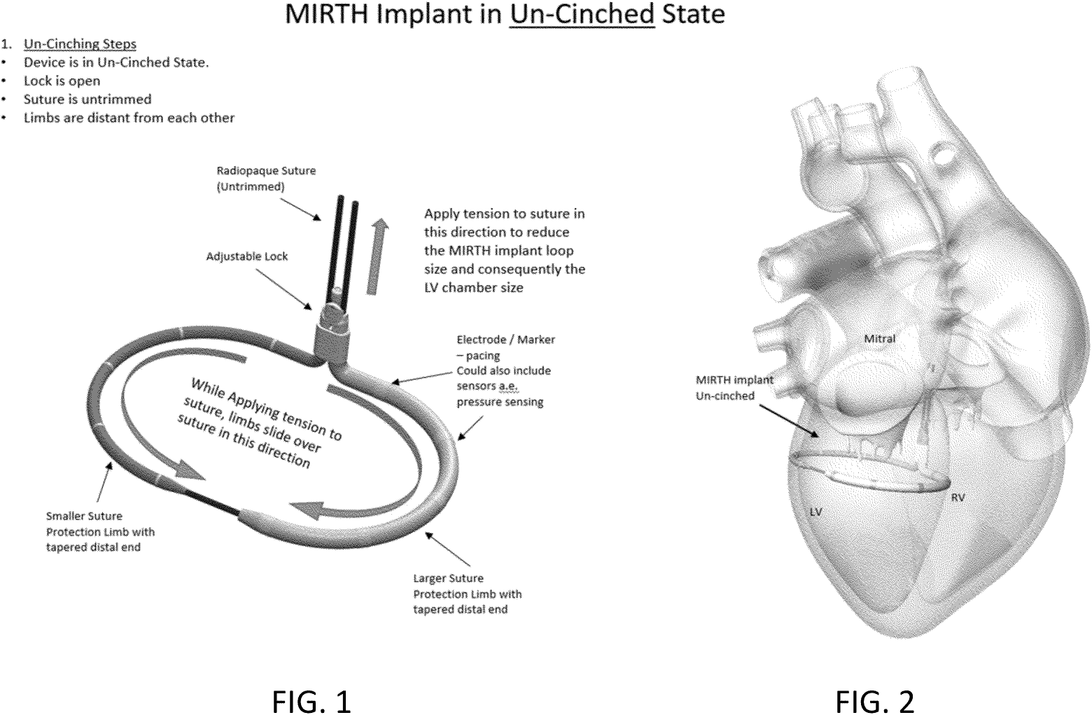

For purposes of illustration, and not limitation, as embodied herein and as illustrated in FIG. 1, an implant designed for use in a MIRTH procedure is depicted. The MIRTH implant includes an elongate flexible tether having two ends (illustrated as being a two-ended “radiopaque suture”) formed into a loop, and an adjustable lock having a lock body disposed over the two ends of the tether. The lock body can be configurable to releasably engaging the elongate flexible tether using a mechanism and lock delivery catheter similar to those illustrated in U.S. Pat. No. 10,433,962. The implant can further include first and second tubular limbs, as illustrated, that extending outwardly (as illustrated, downwardly) from the lock over the elongate flexible tether along the loop toward each other. As illustrated, each limb includes an approximate 90 degree bend shortly after exiting the lock body to facilitate appropriate alignment of the lock and the limbs with respect to the surrounding anatomy. The implant lock can define at least one distal opening therein. As illustrated, the at least one distal opening can be connected to the first and second tubular limbs.

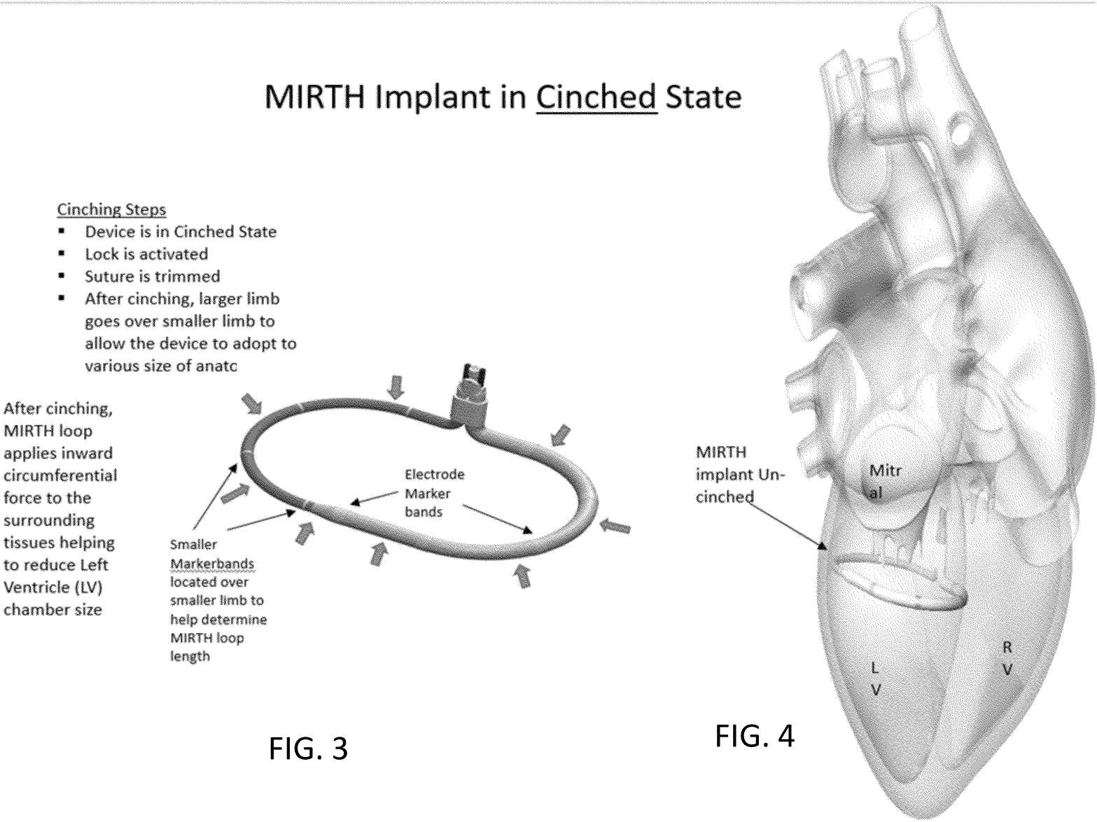

If desired, as illustrated, the first and second tubular limbs can be of different diameters. The first and second tubular limbs can have tapered distal ends, as illustrated in FIG. 1. As illustrated in FIG. 3, the limbs can be advanced along the suture until they contact each other and begin to overlap. As illustrated in FIG. 3, a distal end of a first tubular limb can slide within a distal end of a second tubular limb along the loop of the elongate flexible tether such that the first and second tubular limbs overlap. At least one of the first and second tubular limbs can include a plurality of radiopaque markers along its length. If desired, the inner tether or suture can also be provided with radiopaque markers along its length at regular intervals. For example, FIG. 3 presents marker bands along the smaller diameter limb at left. The plurality of radiopaque markers can be disposed along the length of said at least one of the first and second tubular limbs in a predetermined pattern, as illustrated in order to facilitate measurement of the implant under visualization. This can be done, for example, to estimate the perimeter, or length, of the loop implant upon installation. By additionally or alternatively including radiopaque markers along the inner tether, this can also be accomplished. As will be appreciated, both limbs can include market bands, as desired.

It is useful to include marker bands along different components of any of the implants set forth herein to help judge the relative position of components. But, in further accordance with the disclosure, this can be done as well in order to permit the surgeon to quantify the amount that the implant is contracted, or cinched, when tension is being placed on the tensioning tether during installation. For example, in the instance of the MIRTH implant that is illustrated, the surgeon installing the implant can put the implant in place and introduce the lock with limbs attached over the tensioning tether(s) and into the patient’s heart. When the lock is in position, the relative position of marker bands on the limbs of the lock and/or the inner tether can be noted. Then, as the tensioning tether is tensioned by pulling the tether through the lock and holding the lock in place, the implant decreases in circumferential length, and the marker bands move with respect to each other. Once a predetermined amount of tension is imparted, the implant can be locked, and the amount that the implant has been shortened along a circumferential direction can also be noted. Alternatively, the implant can be contracted by a predetermined circumferential extent simply with reference to the relative positions of the marker bands. Thus, once the operating surgeon notes that a desired amount of distance has been contracted, the lock can be put into place, locking the tether into position.

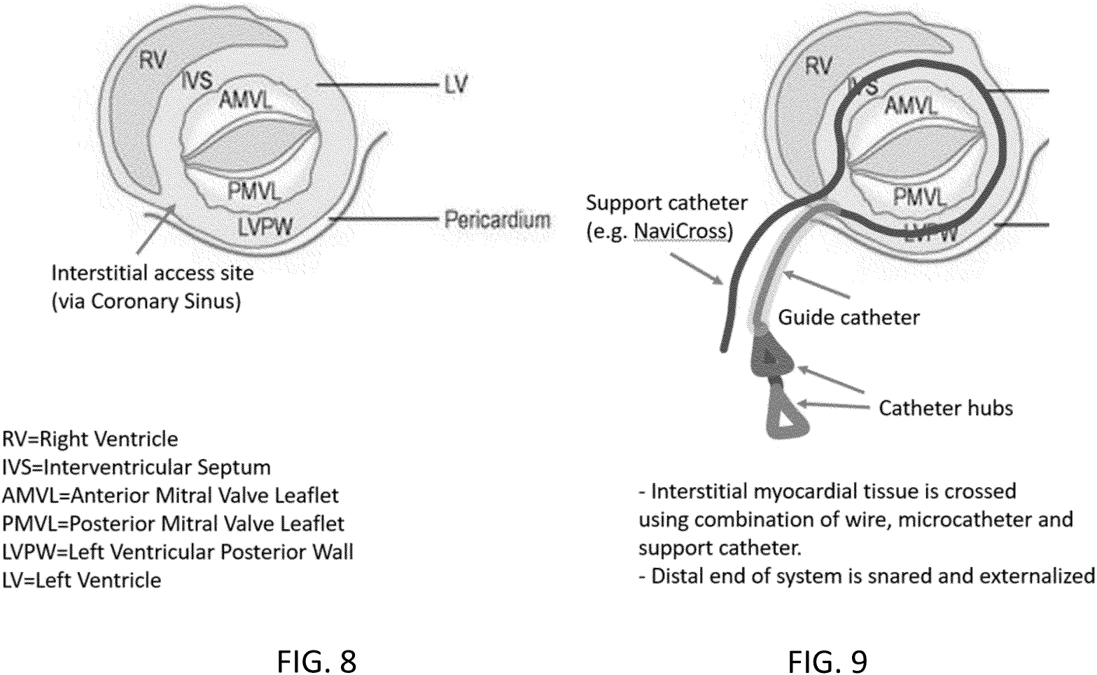

As disclosed herein, the lock joins the free ends of tension element and applies counter-traction to the tension element. Additionally, the lock supplies a dock for delivery and adjustment and can take on a variety of configurations. For example, while the MIRTH implant of FIGS. 1-4 is illustrated as having a wishbone shape in combination with the limbs, this may not necessarily be the case. The lock may slid over a single tether without an extending limb, or perhaps with a single limb, for the embodiment of FIGS. 8-21. Or, the lock may be a crimp, as desired. Similarly, the lock may include an “L”-shaped intramyocardial lock to displace tension from an intramyocardial location to an epicardial or right atrial location. The implant of FIGS. 8-21 can similarly be delivered over one of a pair of tethers, wherein the outer tubular member can be attached to or otherwise pushed over one of the tethers, and both tethers can be threaded through the lock.

As illustrated in FIG. 3, for example, the tubular limb illustrated at right includes a plurality of pacing electrodes formed therein to stimulate cardiac tissue. While not explicitly shown, the implant can further include a controller coupled to the at least one pacing electrode to provide at least one of pacing, defibrillation, measurement and control. For example, the controller can be located in the lock body or may be coupled to the lock body, as desired. If desired, the elongate flexible tether can form an antenna, such as a loop antenna that conducts signals to and from the controller. It will be appreciated that one or more conductors can be embedded in the suture or tether to form an antenna, such as a loop antenna or dipole antenna, for example. Alternatively, the lock body and first and second limbs can be provided with conductive paths that form one or more conductive loops that can act as an antenna when coupled to the controller.

In some embodiments, the pacing device can include a ring electrode array that is effectively implanted deep inside the basal left ventricular myocardium along a MIRTH trajectory to allow synchronous activation (otherwise known as depolarization) of healthy or diseased myocardium in the desired base-to-apex sequence. The ring electrode can include multiple electrodes (e.g., spacing 1, 2 or 3 mm apart) in monopolar or multipolar configurations. The entire array is implanted deep within myocardium, which is not attainable with surgery or with epicardial or endocardial implants.

The problem of right-ventricular pacing-induced dyssynchrony is addressed herein by synchronous depolarization of the entire basal left ventricle. The problem of unreliable (due to location, fibrosis, heterogeneous cardiomyopathy, variable target vein location) capture and resynchronization using standard left ventricular leads is addressed by implanting a deep basal circumferential ring electrode along a MIRTH location. The problem of physical lead insecurity of direct His-bundle pacing electrodes can be addressed using a deep implanted array electrode. The problem of variably high stimulation thresholds of direct His-bundle pacing electrodes can be addressed using a deep intra-myocardial array electrode. The problem of inadvertently inducing tricuspid regurgitation using pacemaker leads can be addressed using a deep MIRTH electrode array. The problem of bulky and insecure defibrillation electrodes that also might cause tricuspid regurgitation can be addressed by using a deep MIRTH electrode array. The problem of endocarditis caused by tissue and valve interaction with conventional pacing/defibrillation leads is mitigated by deep MIRTH electrode arrays.

The implant can additionally or alternatively be provided with a controller and a reservoir (not shown) containing a beneficial agent, wherein the controller can be coupled to a dispenser (not shown) that is coupled to the reservoir to dispense the beneficial agent. The beneficial agent can include one or more of a medication, a gene therapy material, living cells to seed at least one location of the heart that is damaged, and the like. At least one of the first and second tubular limbs can include at least one sensor (not shown) to sense at least one biological parameter. The sensor can include, for example, one or more of a pressure sensor to sense blood pressure, a chemical sensor, a distance sensor, a sensor having circuitry to detect electro physiological data, a movement sensor, and a location sensor.

The elongate flexible tether can include radiopaque material along its length, as desired. The elongate flexible tether can include a hollow braided suture, and the radiopaque material within the elongate flexible tether can include a radiopaque wire that can in turn be disposed within a length of heat shrunk polymeric tube that resides within a hollow core of the elongate inner tether. Additionally or alternatively, the braided suture material can be doped with a radiopaque powdered material in powder form. The implant’s relative orientation surrounding the left ventricle is illustrated in FIG. 4, prior to being cinched.

By way of further example, the inner tether that is used for tensioning the implant and locking it in place can be made from a 1-2 mm ultra high molecular weight polyethylene (“UHMWPE”) coreless round braid from DSM, Dyneema or Teleflex. In some implementations, the tensioning tether can be loaded with at least 20% bismuth by weight to enhance radiopacity. For example, the tensioning tether may be loaded with between about 20 and about 70% bismuth or barium sulfate, or to any degree therebetween in increments of about 1% by weight. Additional or alternative radiopaque materials can be incorporated into the tensioning tether or other portions of the implant or delivery devices or other instruments set forth herein, such as tungsten, tantalum, and barium sulfate. These materials can be incorporated, for example, as drawn metallic (e.g., platinum, or other radiopaque material) wires incorporated into the braiding, such as by weaving, or by directing the drawn wire along a central channel defined within the tether. In a further embodiment, ultra high molecular weight polyethylene can be used as a tensioning tether material for improved creep resistance, and is preferably 1-2 mm in size, and is commercially available from Teleflex Corporation. While braided materials are illustrated for the tensioning tether, it will be appreciated that any other suitable material can be used.

In a further implementation, the tensioning element can include a braided suture as described herein, or other outer tubular member, provided with a core member that includes a radiopaque core disposed within the outer tubular member. This can include a solid or braided wire or cable including radiopaque material, or a smaller braided tether, for example, that is doped or otherwise modified to include bismuth or other radiopaque material.

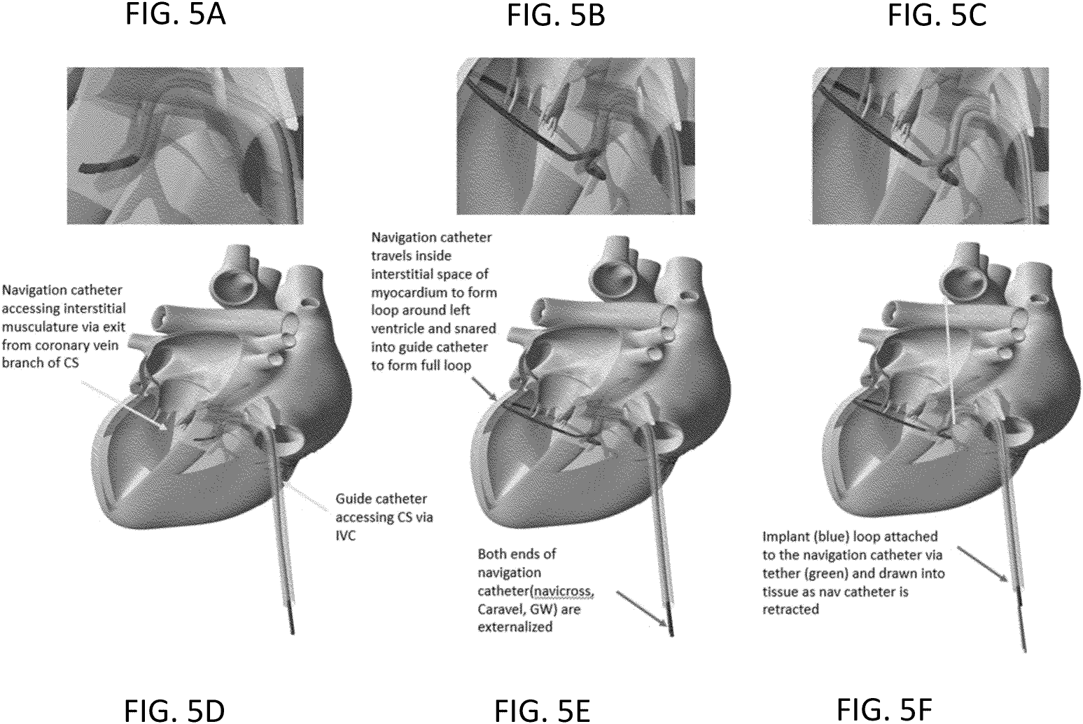

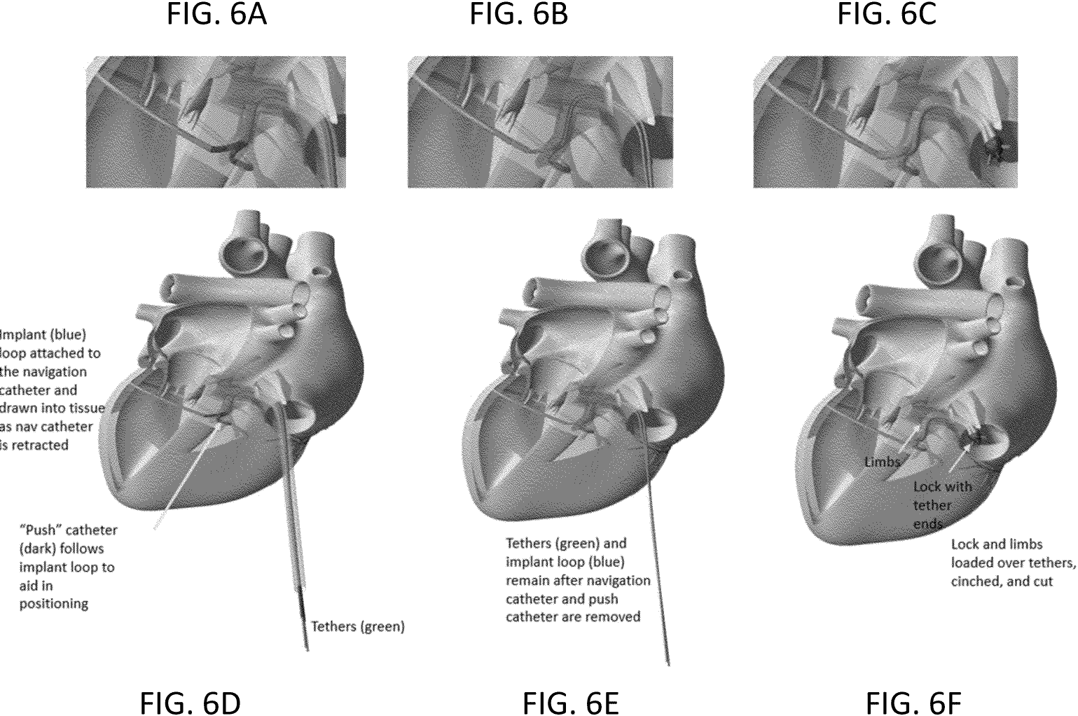

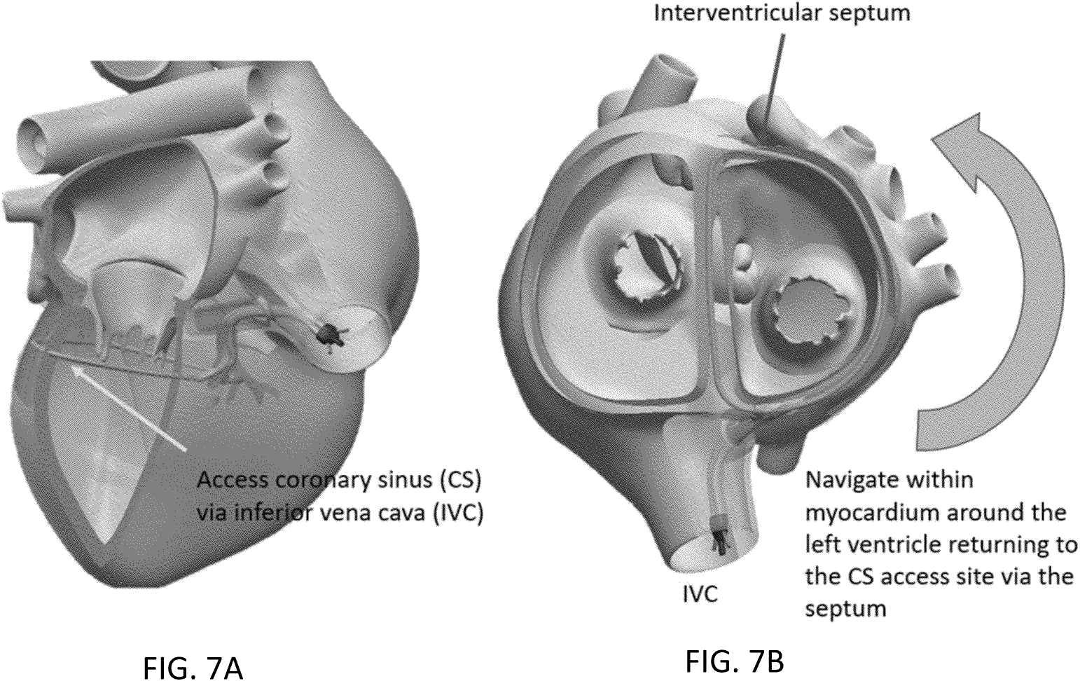

To review the installation steps of the illustrated implant, reference is herein made to FIGS. 5A-7B. FIG. 5A shows a closeup of the image of 5D, which illustrates a guide catheter accessing the coronary sinus by way of the Inferior Vena Cava (IVC). A navigation catheter then accesses interstitial musculature by way of an exit from the coronary branch of the coronary sinus. FIGS. 5B and 5E cooperate to illustrate the navigation catheter, which is disposed in the guiding catheter, traveling inside interstitial space of the myocardium to form a loop around the left ventricle. The navigation catheter is then snared into a snare catheter and withdrawn into the guide catheter to form a full loop. Both ends of the navigation catheter are then externalized. A retention tether that passes through the navigation catheter, which is now externalized, is attached to a distal end of the flexible tether portion of the MIRTH implant. The MIRTH implant is then pulled into the anatomy surrounding the LV until both ends of the flexible tether portion of the MIRTH implant are externalized. A push catheter can be used to help push the MIRTH implant into place. The MIRTH implant, in this illustration including a flexible body with a tether extending from each end, has both tether ends externalized to permit the lock with attached limbs to be threaded over the two ends of the MIRTH implant. The MIRTH implant can include, for example, a flexible loop made from a hollow braided suture with a thickened portion in the middle that sits in the ventricle of increased diameter. This can be done, for example, by inserting a solid flexible body, such as a tubular member into the hollow tether in order to thicken it, or by a variety of other techniques as discussed below. As such, the flexible portion of the MIRTH implant can be formed from a continuous hollow suture with a thickened central portion, wherein the portions of the tether that extend beyond the lock can be empty or have minimal structural elements within them, such as conductive wires, and the like, so they can be threaded through the lock and cut off near the lock after the lock is installed. Thus, as shown in FIGS. 5-7, the MIRTH implant is progressively introduced once the passageway is created by a guidewire (not shown) and a navigation catheter, and support catheter, if required, inside of a guiding catheter, resulting in the final installation depicted in FIG. 7A and FIG. 7B.

FIGS. 8-21 depict a further implant and associated installation method for performing a RAMIN procedure. This type of implant and installation can be used, for example, for a MIRTH or SCIMITAR procedure. A detailed description follows later concerning specific illustrative steps that can be used in order to establish the guidewire trajectory to create the passageway for the implants as discussed herein, so they will not be discussed in detail here.

But, once guidewire trajectory is established, the tension element or implant has to be delivered and deployed, and then must shorten to alter the geometry of the targeted myocardium. The myocardial tissue creates resistance to delivery which requires a combination of column strength, tension, and lubricity. Once delivered, the tension element is preferably capable of being manipulated to permit it to expand its diameter so as to reduce erosion or “cheesecutting”, and to shorten in length. If desired, the support catheter(s) used to help expand the passageway defined by the guidewire can be enhanced with a fluid exit port that can inject saline to create a locally pressurized zone to effect hydrodissection to help bluntly dissect the myocardium, and also to add lubricity to assist in passageway expansion.

As to thickness of the dissecting element, starting with a guidewire of 0.014-0.035 inches, the passageway is preferably then dilated to accommodate an implant having a thickness of 2-3 mm. Preferably the implant itself has length markers (e.g., radiopaque markers). Similarly, the diameter of the implant can be increased upon implantation, as discussed below. Having an implant to prevent erosion and pull through and myocardial laceration is of particular concern around curves such as septal-posterior SCIMITAR and anterior-reentry in SCIMITAR during implant delivery into a tortuous myocardial trajectory.

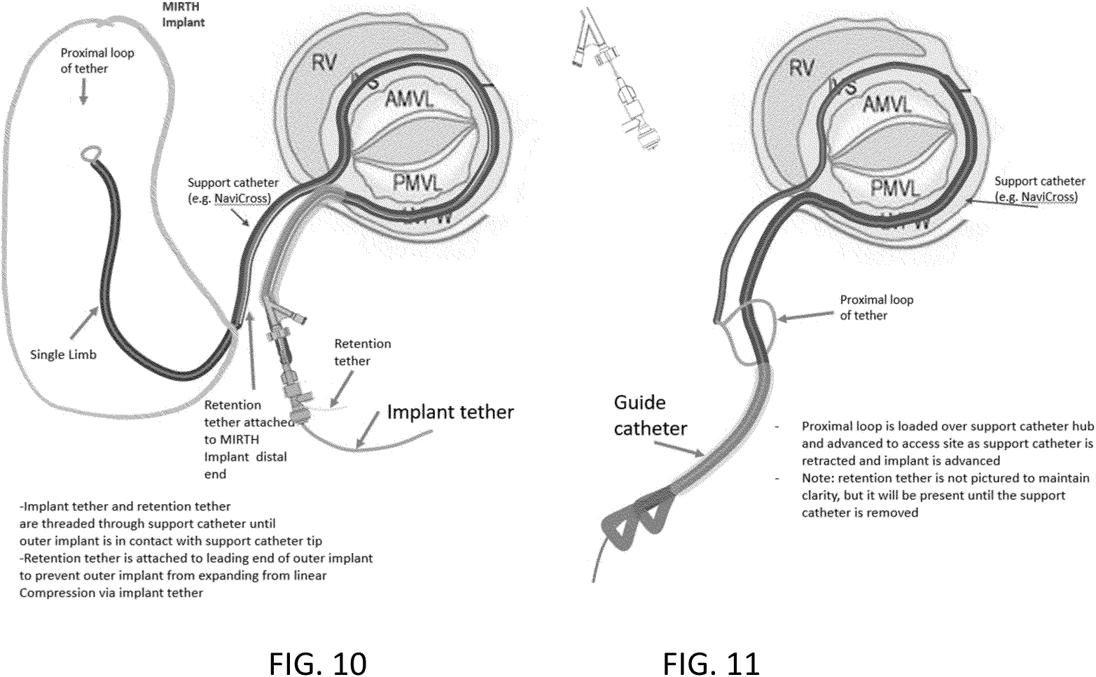

For purposes of illustration, and not limitation, FIG. 10 presents an implant that includes an elongate inner tether having a proximal end and a distal end. The proximal end of the elongate inner tether can terminate in a loop, as illustrated. The implant can include an outer tubular body (indicated in FIGS. 10 and 11 as a thickened line surrounding the looped tether) that surrounds the elongate inner tether along at least a portion of the length of the inner tether. The outer tubular body can be expected to be shorter in length than the elongate inner tether. As set forth below, the illustrated implant can be configured to shorten in length and increase in transverse dimension when it is compressed along an axial direction.

As illustrated in FIGS. 8-15, the associated procedure of installing the illustrated implant will typically begin with a dissection process as disclosed herein to define a passageway to receive the implant, such as by using one or more support catheters. FIGS. 8 and 9 illustrate the passageway defined by a support catheter (e.g., Navicross) that supports and surrounds an electrified guidewire or other dissection catheter, discussed in further detail below. The distal end of the guidewire and support catheter are captured and externalized, as illustrated in FIG. 9.

As illustrated in FIGS. 10-11, the distal end of the tensioning tether inside of the outer tubular member of the implant is then withdrawn through the support catheter, for example, by simply attaching the tensioning tether to the guidewire that is still residing in the support catheter. The guidewire is then withdrawn through the support catheter, pulling along the tensioning tether of the implant with it. If desired, along with the tensioning tether, which forms the core of the implant, a distal end of a “retention” tether can also be attached to the guidewire and withdrawn through the support catheter along with the tensioning tether. A proximal end of the retention tether can be attached to a distal end of the outer tubular member. This permits the tensioning tether to be withdrawn, into the supporting catheter. But, the retention catheter pulls on the distal end of the outer tubular member, thereby pulling it into the patient’s vasculature as the supporting catheter is withdrawn. This prevents the need for the tensioning tether to be used to pull the outer tubular member, which if so configured could cause the outer tubular member to shorten and expand. The outer tubular member can then be expanded at a later time by pulling on the tensioning tether, but not while the outer tubular member is being transported into the heart.

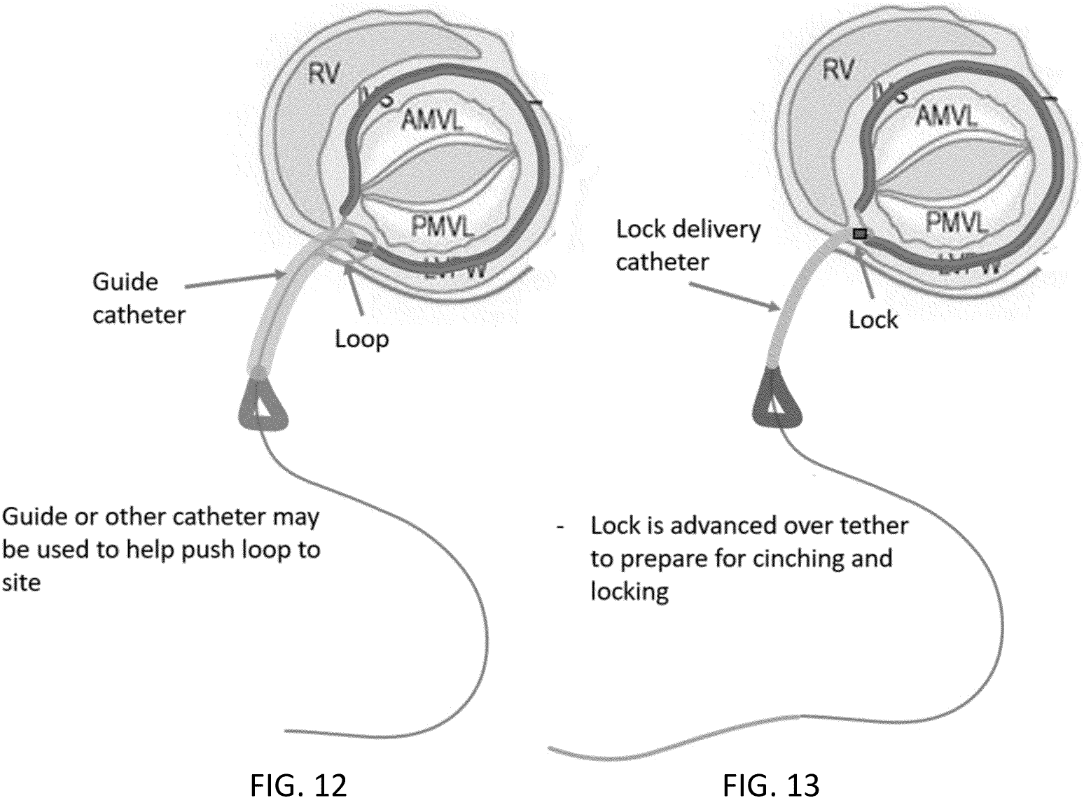

Thus, by virtue principally of tension imparted by the retention catheter, the outer tubular member can then be pulled to butt up against the distal end of the externalized support catheter, and the support catheter can be withdrawn, pulling the outer tubular member of the implant along with it. As illustrated in FIG. 11, the distal end of the tensioning tether can be directed through a proximal loop formed by its own proximal end. FIGS. 12 and 13 illustrate the loop of the tensioning tether being pulled into the heart. At this time the implant can be cinched by threading a lock delivery catheter over the single tensioning tether, that acts as a rail, as illustrated in FIG. 13 and FIG. 14. As illustrated in FIG. 15, the excess tether can be cut, using a cutting catheter, for example, as described in U.S. Pat. No. 10,433,962.

It will further be appreciated that the inner tensioning tether of the implant embodiment of FIG. 1 can similarly be provided with a proximal loop that when externalized is looped over the proximal end of the catheter that is used to deliver the implant and apply tension so that tension is being applied to one tether end through a lock body that has a single tether running through it. Moreover, it will be appreciated that a lock body can similarly be advanced over the single tensioning tether illustrated in FIGS. 12 and 13.

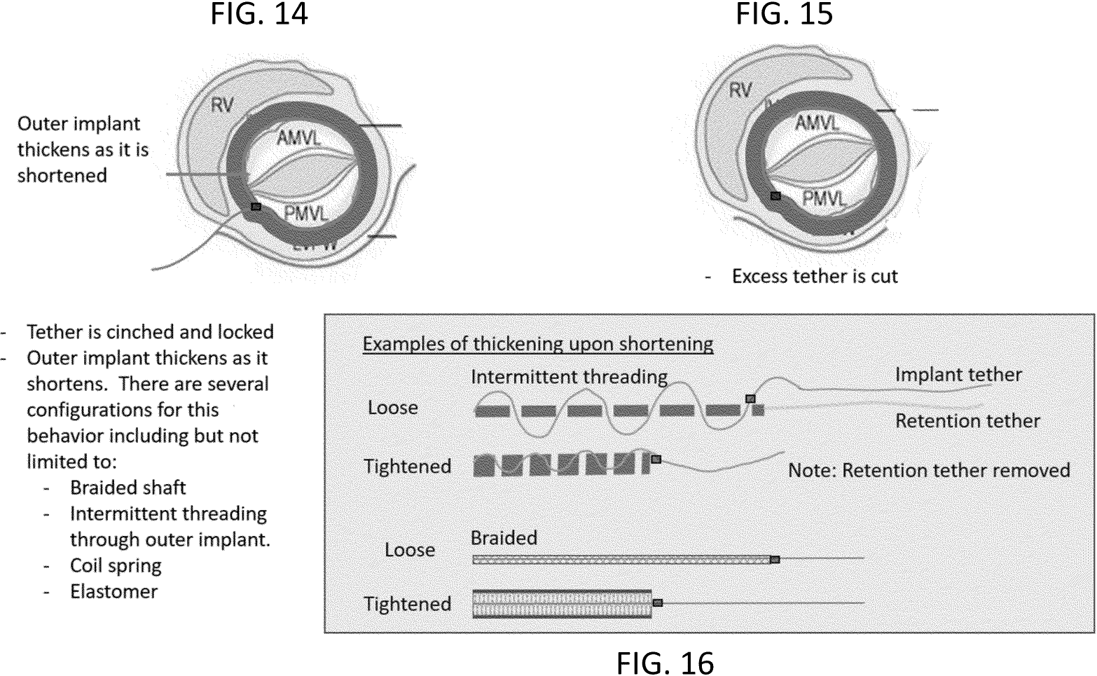

Various arrangements can be used to cause the outer tubular member to expand in diameter after it is placed in the passageway through the heart, or other anatomy. For example, as illustrated in FIG. 16, the tensioning tether (illustrated as “implant tether”) can be threaded in and out of the implant loosely for when the outer tubular member is being introduced. The retention tether is illustrated as being coupled to the distal end of the outer tubular member. The tether can then be tensioned after the outer tubular member has been positioned.

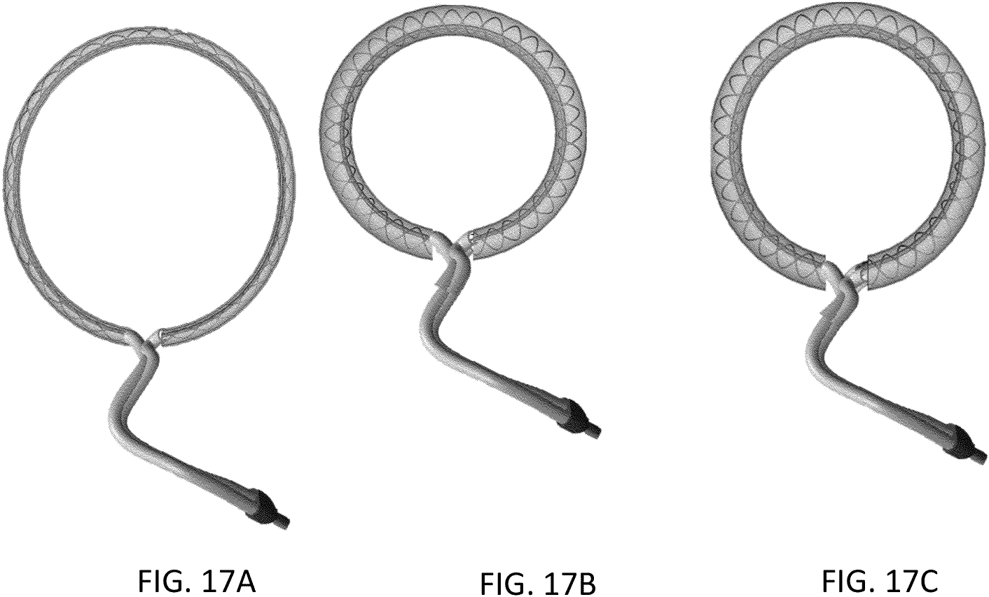

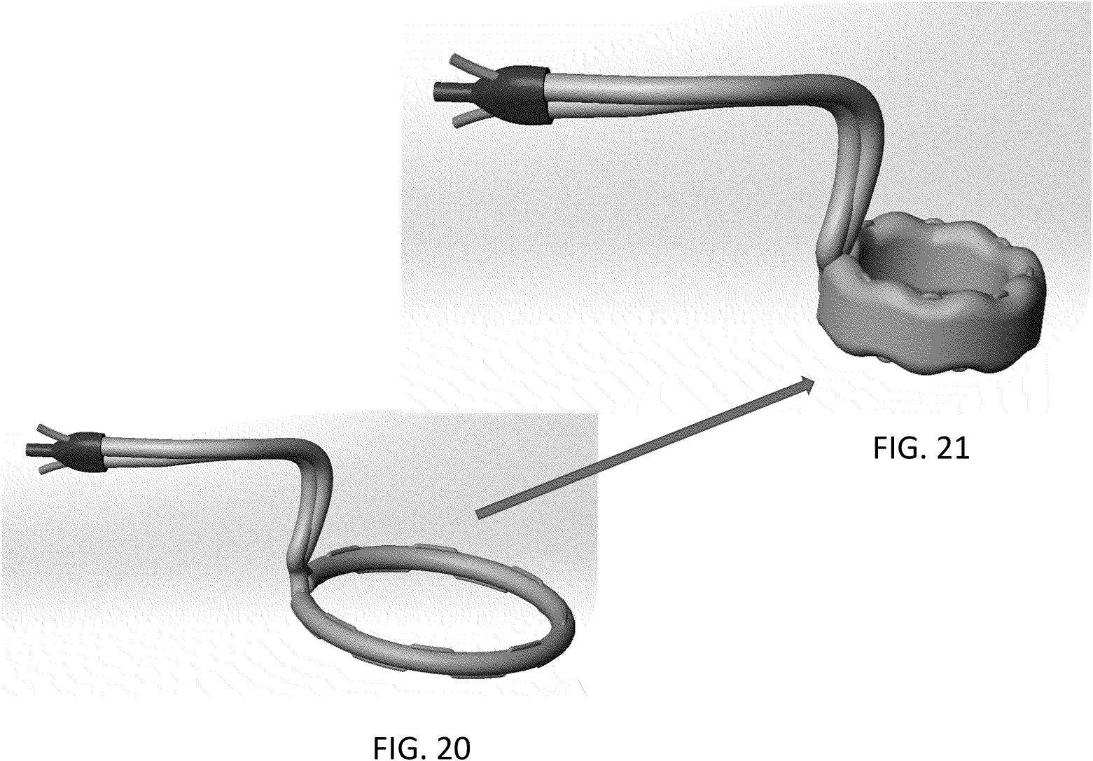

As show in FIGS. 17A-17C, the outer tubular body can include a braided structure. In some implementations as illustrated in FIGS. 16, 20 and 21, the elongate inner tether can be threaded intermittently through the outer tubular body. More specifically, as can be seen, the elongate inner tether is first directed through the implant across its width, then along one surface of the implant, then directed through the implant across its width again along an opposite direction, and then directed along the implant, through the implant, and so on. As illustrated, this pattern of the tether can form the shape of a square wave, for example, or can be sinusoidal, or have a sawtooth shape. This can permit the implant to be compliant and can permit the implant to change in length in response to movements of the heart. Specifically, it will be appreciated that since the elongate inner tether is routed in a manner where it crosses the width of the implant repeatedly, the length of the tether that passes through the implant body is actually considerably longer in length than the implant body. Thus, with reference to FIG. 20, the implant is illustrated in a condition wherein the implant body is at an uncontracted length. When the tether is tightened, the length of the implant is reduced, and its thickness is increased, as illustrated in FIG. 21. A variety of shapes can be created along the length of the implant that is facilitated by the manner in which the tether is threaded through the implant body, as well as the stiffness of the implant body. For example, the implant body can be formed from a multilayered structure that includes materials of different durometers, and/or thicknesses along the length of the implant body. Braiding of metallic or other filaments can form a braided layer, wherein the braiding can be denser in regions that are not intended to flex as much as areas that have less braiding, for example. The regions of more or less braiding, or areas of more or less rigidity of the implant generally can be aligned with the routing of the tether to facilitate collapse of the implant along an axial direction, and expansion of the implant along a radial direction. Even when tightened, the tether will still be longer than the implant body, and the compressibility and flexibility of the implant body can permit the installed implant to flex and adjust in effective length to accommodate the beating of the heart. If desired, the implant body can be made from adjacent regions of different stiffness as well, such as from a flexible inner layer of material that includes discrete lengths of stiffer material around the inner layer that are separated from one another in a manner resembling beads on a string. Contracting the length of the implant can cause the regions of stiffer material to move toward one another, compressing the flexible inner layer of material between them.

In some implementations, the outer tubular body can include a resilient member. If desired, the outer tubular body can include a shape memory material, a resilient member, and/or a coil spring. In some implementations, the outer tubular body can include a plurality of radiopaque markers along its length. The plurality of radiopaque markers disposed along the length of the outer tubular body can be arranged at predetermined intervals to facilitate measurement of the implant under visualization.

As with the MIRTH implant, the outer tubular body of the implant of FIGS. 8-21 can include at least one pacing electrode to stimulate cardiac tissue. A discrete number of combined pacing electrodes can be used, such as 4 or 8 or 12 around the circumference of the heart, or a portion of the heart, to resynchronize the heart when there is dyssynchronous contraction caused by conduction system disease, particularly in the setting of ischemic or non-ischemic cardiomyopathy. If desired, the implant can further include a controller (embedded in the lock body, for example), coupled to the at least one pacing electrode to provide at least one of pacing, defibrillation, measurement and control. The implant of FIGS. 8-21 can include an antenna, such as a loop antenna, dipole antenna, monopole antenna, helix antenna, and the like, that conducts signals to and from the controller. If desired, the implant of FIGS. 8-21 can include a controller and a reservoir (not shown) containing a beneficial agent. The controller can be coupled to a dispenser (not shown) that is coupled to the reservoir to dispense the beneficial agent. If desired, the beneficial agent can include one or more of a medication, a gene therapy material, and living cells to seed at least one location of the heart that is damaged. If desired, the outer tubular body of the implant of FIGS. 8-21 can include at least one sensor to sense at least one biological parameter. At least one sensor can include at least one pressure sensor to sense blood pressure. The at least one sensor can include at least one of: a chemical sensor, a distance sensor, a sensor having circuitry to detect electro physiological data, a movement sensor, and a location sensor. The elongate inner tether can be a hollow braided suture, and the radiopaque material within the elongate inner tether can include a radiopaque wire disposed within a length of heat shrunk polymeric tube that resides within a hollow core of the elongate inner tether.

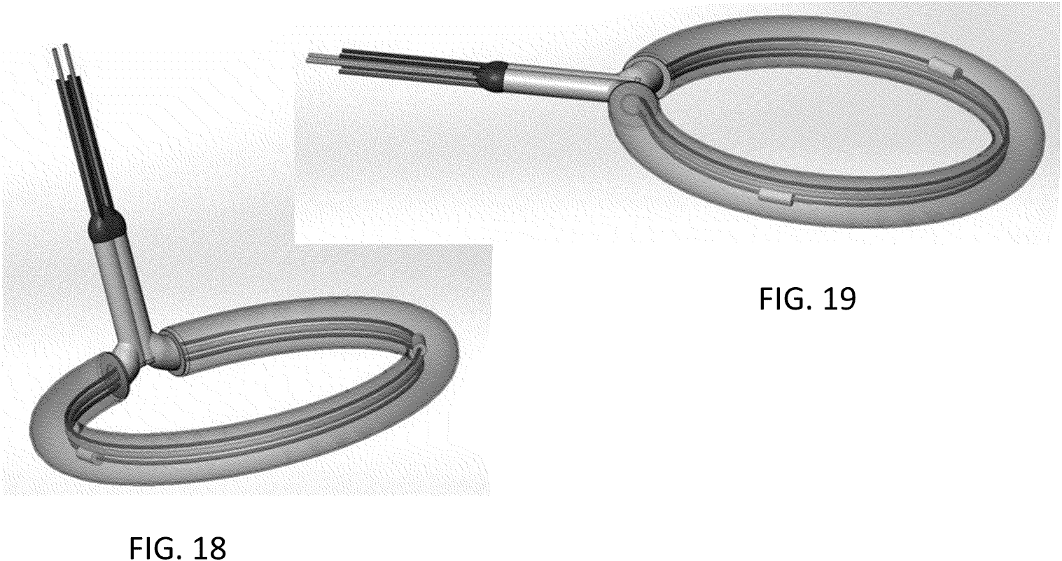

As illustrated in FIGS. 18 and 19, an implant is depicted that shortens in length and thickens when tension is applied to it that includes electrodes and marker bands disposed along the inner tensioning tether portion of the implant. As can be seen, circuit paths traverse the loop path of the inner tether and come out through the lock body, and the circuits can be completed, if desired, by actuating the lock, or the circuitry can be complete within the implant, and the lock can simply hold the implant together.

In further accordance with the disclosure, the durometer of the limbs and outer tubular members, and inner tensioning member can be varied along their length. For example, for a portion of a limb that is expected to be present in the septum of a patent rather than an outer wall of the heart can be provided with a different stiffness than the portion in the outer wall of the heart. The change in durometer can be accomplished by varying the type of material or thickness of material along the length of the implant. Additionally or alternatively, the durometer can be varied by the manner in which the material of the implant collapses when tension is applied to the tensioning tether. For example, the spacing of undulations of the tensioning tether in FIG. 16 can be varied along the length of the implant so that the frequency of stitching per unit length of the un-cinched outer tubular member is different in different regions of the outer tubular member.

The implant can further include an implant lock configured to lock the implant into a loop form. The act of locking the lock, such as by engaging the lock, can complete an electrical circuit to permit any of the sensors or pacing devices to be activated and/or used. For example, the inner tensioning tether can include a variety of conductors and the like that have an electrically insulating layer that is penetrated, for example, by a sharp barb within the lock body when the implant is locked in place by putting pressure on the tensioning tether. Putting pressure onto the conductor during the locking process can result in a protrusion or barb within the lock body penetrating an insulating layer within the tether to complete a circuit.

In some implementations, advancing the guidewire through the myocardium when practicing a procedure as set forth herein can include ablating tissue. For example, the myocardial tissue can be ablated by applying electrical energy through a guidewire to energize an electrically uninsulated exposed distal end surface of the guidewire. This is typically accompanied by advancing a supporting catheter over the guidewire to provide additional column strength to the guidewire. The distal end portion of the guidewire can include at least one visually enhanced marker visible under a visualization mode. Related methods can include visualizing the guidewire and myocardium under the visualization mode during the procedure to help control advancement of the guidewire through the myocardial tissue.

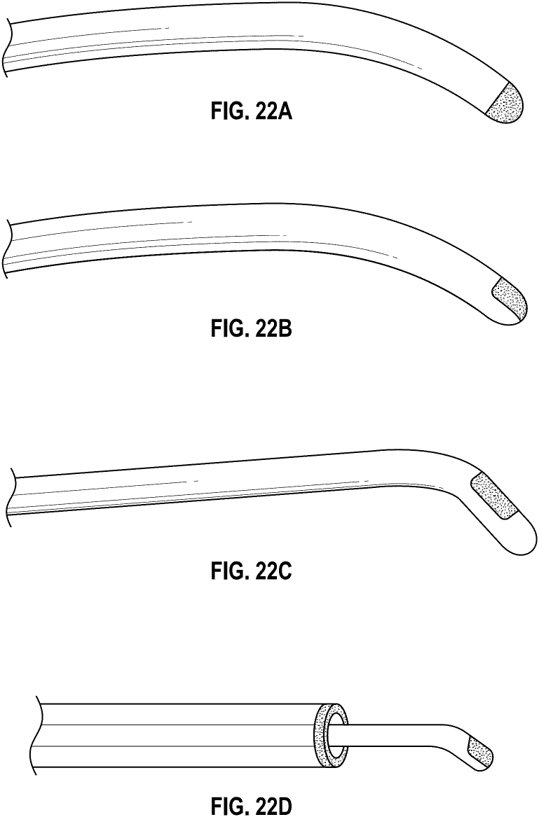

For purposes of illustration, and not limitation, FIGS. 22A-22D depict schematic views of illustrative guidewire tips in accordance with the present disclosure. These guidewires can be used for myocardial navigation and traversal. Once introduced into the myocardium, these guidewires can steer and advance throughout heart muscle to create a range of desirable trajectories in order to introduce other devices. This permits conformal electrosurgical advancement when tissue planes do not readily allow traversal. Unipolar or bipolar RF power can be applied at a range of frequencies. However, in further accordance with the disclosure, these guidewires can also permit recording and monitoring of intracardiac electrograms to guide navigation, because different intramyocardial locations (endocardial to epicardial) exhibit characteristic unipolar and bipolar electrograms.

Any of the depicted guidewires can be included with an elongate passage, such as by using a hyptotube, to permit hydrodissection gas-dissection at or near the distal tip of the guidewire, or to inject a different beneficial agent. As depicted, the guidewires in FIGS. 22A-22D can be provided with asymmetric insulation to create conformal electromagnetic fields inside myocardium. The guidewires preferably conform to a form factor of 0.014 inch diameter, and have a steerable fixed CTO curve, and are electrically insulated except for the proximal end region (e.g., 10 mm) and a distal region of about 1 mm. The tip can be symmetrical as depicted in FIG. 22A (darkened end point), but also be bent to permit steering. The exposed patch can be on one side of the distal tip, as in FIG. 22B, or the asymmetric exposed patch can be located immediately proximal of the distal tip as depicted in FIG. 22C. FIG. 22D presents a schematic that combines the embodiment of FIG. 22B with a standalone 0.014” microcatheter having a distal monopole for electrosurgical ablation. These guidewires of FIGS. 22A-22C can be combined into a kit with detachable connector to standard electrosurgery generator that allows activation of the “cut” button for continuous duty cycle RF ablation at selected operational settings.

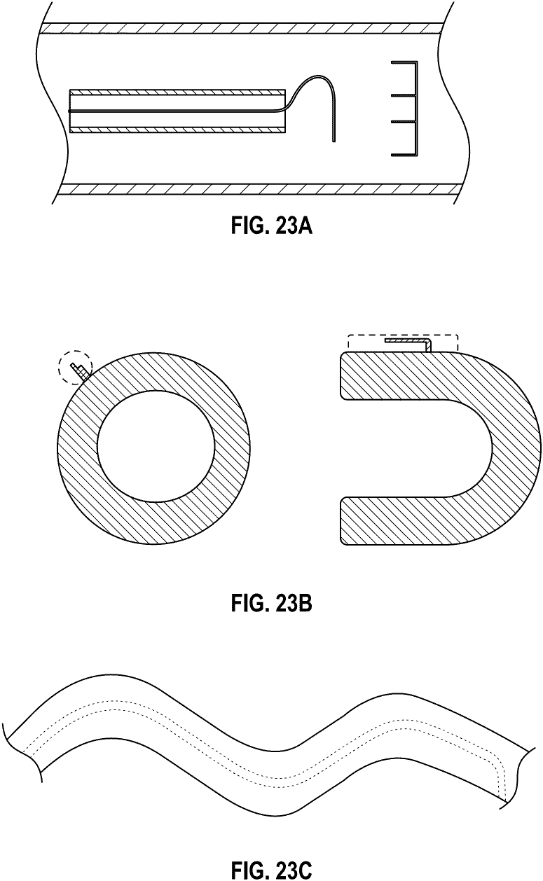

To access the myocardium, it is typically necessary for a guidewire to proceed laterally into tissue after having traversed a longitudinal direction. A centripetal access catheter can be used to provide this initial access into the myocardium. A schematic of an example of an illustrative, non-limiting embodiment of such an instrument is depicted in FIGS. 23A-23C. Functionally, this catheter is used by advancing it over the guidewire to a target location so that the distal tip of the guidewire is steered sharply into the myocardium. Thus, the centripetal access catheter can be made from a polymeric tubular member with a side access port to redirect the guidewire. This can also be accomplished by having a centripetal access catheter that defines a serpentine tip that directs the guidewire first away from the myocardium, and then guides the guidewire through a 180 degree path so that it enters the myocardium directly. The centripetal accessor catheter can include a radiopaque marker near its distal end that indicates the relative rotational position of the centripetal accessor catheter so that the surgeon knows when the correct orientation of the guidewire has been obtained to enter the myocardium. Preferably, the lumen of the centripetal access catheter is lubricious and electrically insulated, and its outer surface is also lubricious to navigate coronary veins. If desired, an inflatable element can be provided along a side of the guiding catheter opposite the guidewire exit port so as to provide a counteracting force against an opposing side of the lumen. The centripetal access catheter is therefore preferably advanced in tandem with the electrosurgical guidewire. An angiography port can be provided to aid in anatomic localization, and the centripetal access catheter is preferably provided with a small profile (e.g., 6-8 Fr) so it can also fit into a larger guiding catheter, such as a coronary sinus balloon-tip guiding sheath. FIG. 23A depicts a sketch of the guidewire path following a switchback path that directs it into the myocardium. FIG. 23B depicts orientation of the centripetal catheter (end view) with respect to the LV, and also a side view of the catheter with the LV viewed in the long axis. FIG. 23C depicts a cross section of an example of the catheter, having a guidewire exit port on its lateral side.

The disclosure further provides a catheter that includes an elongate tubular member coupled to an inflatable member near a distal end of the catheter and a reservoir of inflation fluid, and a collapsible snare surrounding the inflatable member, wherein inflation of the inflatable member with inflation fluid causes the collapsible snare to expand. The collapsible snare is a single loop snare. The collapsible snare can be a multiple loop snare. The collapsible snare can be configured to remain open after the inflatable member is deflated.

Thus, a method is also provided that includes disposing a distal end of a guidewire using a snare catheter at a target location, wherein the snare catheter includes an inflatable member disposed inside the snare, and further wherein inflation of the inflatable member causes the snare to expand. This can be done to bluntly dissect surrounding tissue to make room for the snare. The balloon can be deflated after the dissection occurs, and the snare catheter can then capture the guidewire and collapse to trap the guidewire. For example, this guidewire capturing step can occur in the myocardium. It can similarly be accomplished outside of the myocardium. The elongate passageway can be formed at least in part by directing a pressurized fluid to a target location within the myocardium.

The disclosure still further provides embodiments of a snare catheter that includes an elongate core member having a proximal end and a distal end, an elongate intermediate tubular member having a proximal end, a distal end and defining an elongate lumen therethrough for slidably receiving the elongate core member therein, a collapsible tubular perforated body formed from a plurality of braided members, for example, attached at a proximal end thereof to the distal end of the elongate intermediate tubular member, and at a distal end thereof to the distal end of the elongate core member, wherein relative axial displacement of the distal end of the elongate intermediate tubular member toward the distal end of the elongate core member causes the collapsible tubular perforated body to expand radially outwardly and for the braided members to mutually separate, and relative axial displacement of the distal end of the elongate intermediate tubular member away from the distal end of the elongate core member causes the collapsible tubular perforated body to collapse radially inwardly and for the braided members to collapse together. The snare catheter can further include a target wire disposed within the collapsible tubular perforated body that extends along the elongate core member and has a proximal end attached to the elongate intermediate tubular member and a distal end attached to the elongate core member. The target wire can be configured to assume a first generally straight configuration when the collapsible tubular perforated body is collapsed radially inwardly, and a second substantially nonlinear configuration when the collapsible tubular perforated body is expanded radially outwardly. The snare catheter can further include an elongate tubular longitudinally displaceable sheath having a proximal end, a distal end and defining an elongate lumen therethrough for slidably receiving the elongate core member, elongate intermediate tubular member, collapsible tubular perforated body, and target wire therein when the collapsible tubular perforated body is in a generally radially collapsed state. Particular embodiments of such snare catheters are set forth, for example, in U.S. Pat. No. 10,433,962.

If desired, the elongate core member of the snare catheter can be a tubular member defining a guidewire lumen therethrough. The snare catheter can be provided with an atraumatic distal tip formed from compliant material that is attached to the distal end of the elongate core member. The snare catheter (or any device described herein) can further include radiopaque marker bands disposed near the distal end of the catheter and the distal end of the elongate intermediate tubular member. If desired, the snare catheter can include a plurality of radiopaque marker bands formed on the target wire. The target wire can be formed at least in part from radiopaque material. The collapsible tubular perforated body can be formed at least in part from radiopaque material.

In some implementations, the target wire can include at least one loop and/or undulation formed therein when it is longitudinally contracted. If desired, the target wire can include a plurality of loops and/or undulations formed therein when it is longitudinally contracted. The target wire and loop (and/or undulation) can substantially lay in a single plane parallel to a longitudinal axis of the catheter when the target wire is longitudinally contracted. The target wire and loop(s) and/or undulation(s) can define a three dimensional geometry when the target wire is longitudinally contracted. If desired, a plurality of target wires can be provided having one or more loops and/or undulations when the target wires are longitudinally contracted. The target wire can include composite wire, such as a wire that includes a core portion made from a first material, and a cladding portion made from a second material different from the first material.

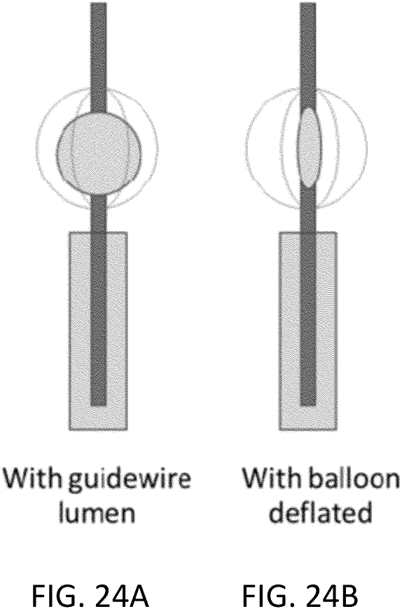

For purposes of illustration, and not limitation, a balloon snare catheter is provided in FIGS. 24A-24B to permit reentry with the disclosed guidewires once they return to form a loop, and to allow recovery, externalization, and exchange of such guidewires for implant components, all while the snare and the guidewire tip are buried inside myocardium.

As will be appreciated, it is difficult to capture a guidewire inside of a path inside of tensioned tissue inside of a beating heart. This issue can be addressed by creating space using a balloon-expandable snare. This catheter can be used to create intramyocardial empty space by balloon-expansion to open a snare, followed by deflation of the balloon while keeping the snare open, to create a space that can be entered by the guidewire, followed by collapse of the snare to withdraw and externalize the guidewire tip. The snare can include a single or multi loop design. The snare can include a plurality of straight elements that expand into the general shape of a sphere or other open volume. The snare can alternatively include a helical wire that when elongated is generally straight, and when compressed, such as by rotating and/or pushing the two ends of the wire together, cause it to expand radially to create a space to permit capturing of the distal tip of the guidewire.

### Example

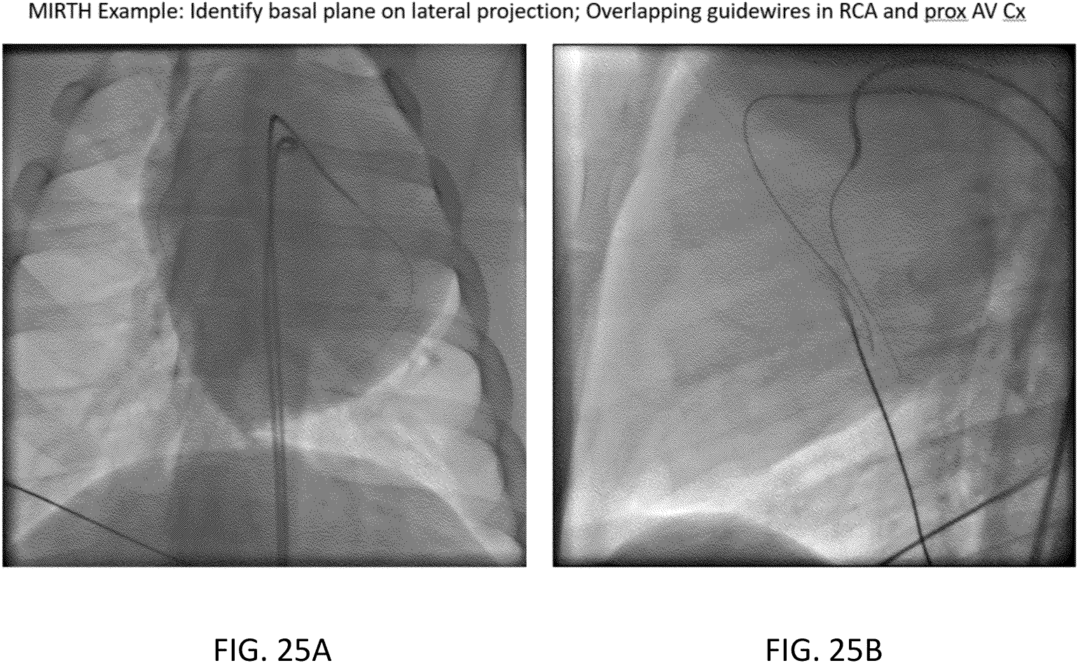

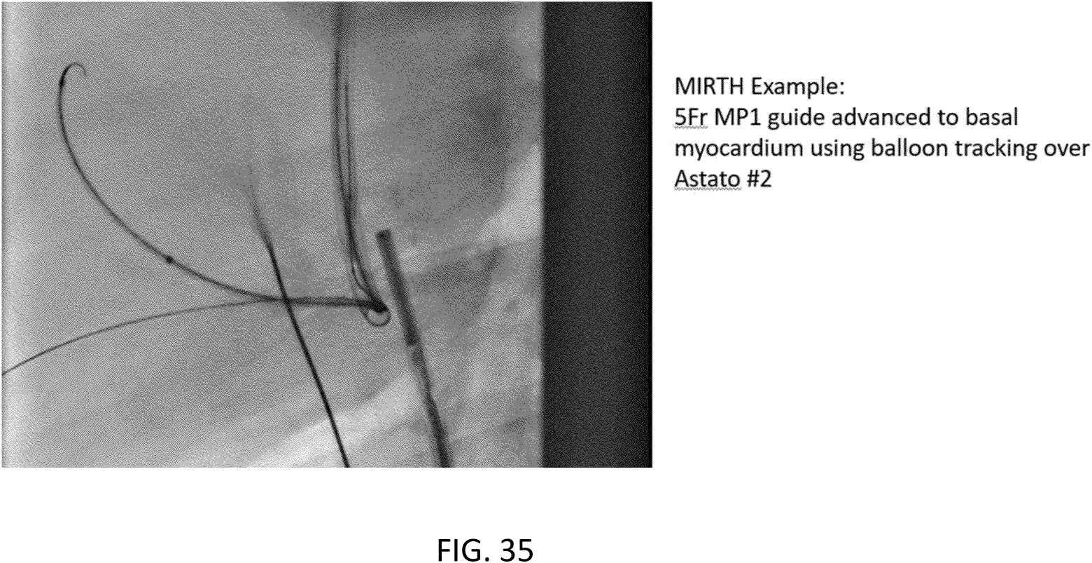

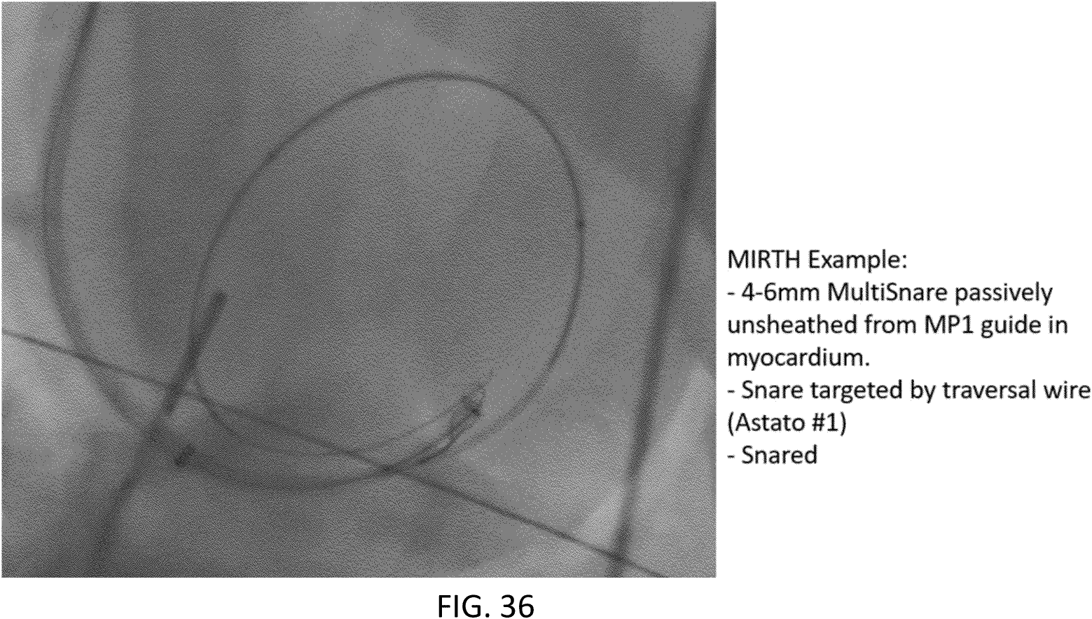

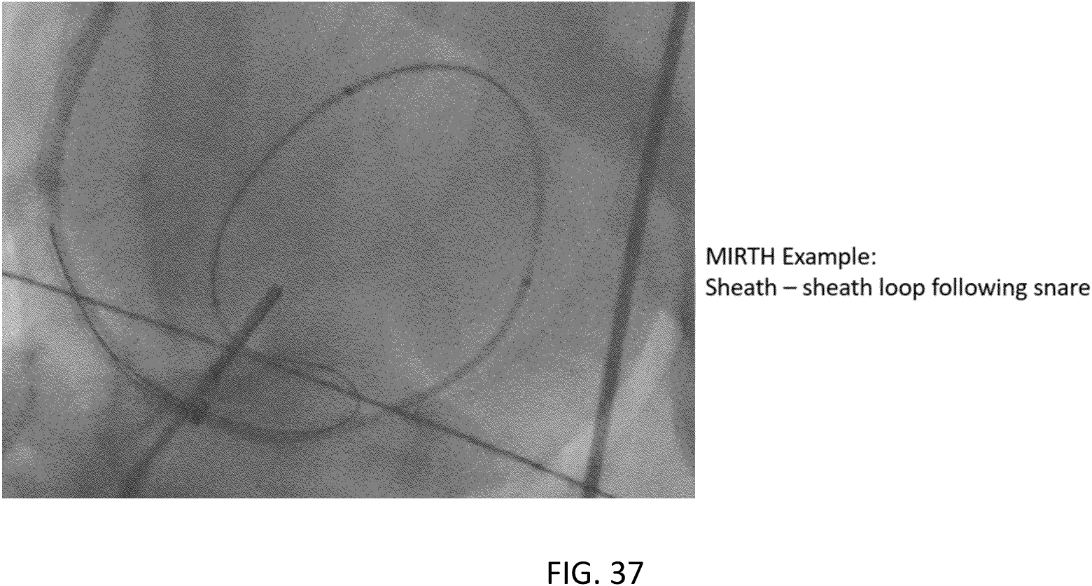

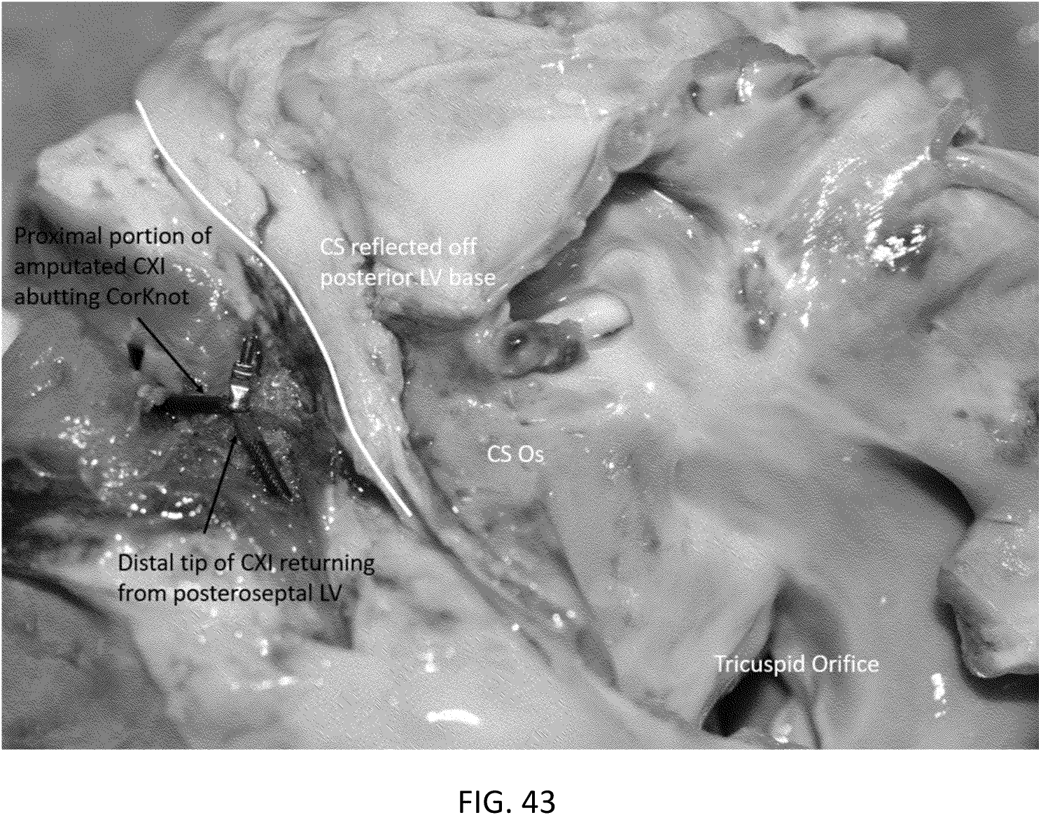

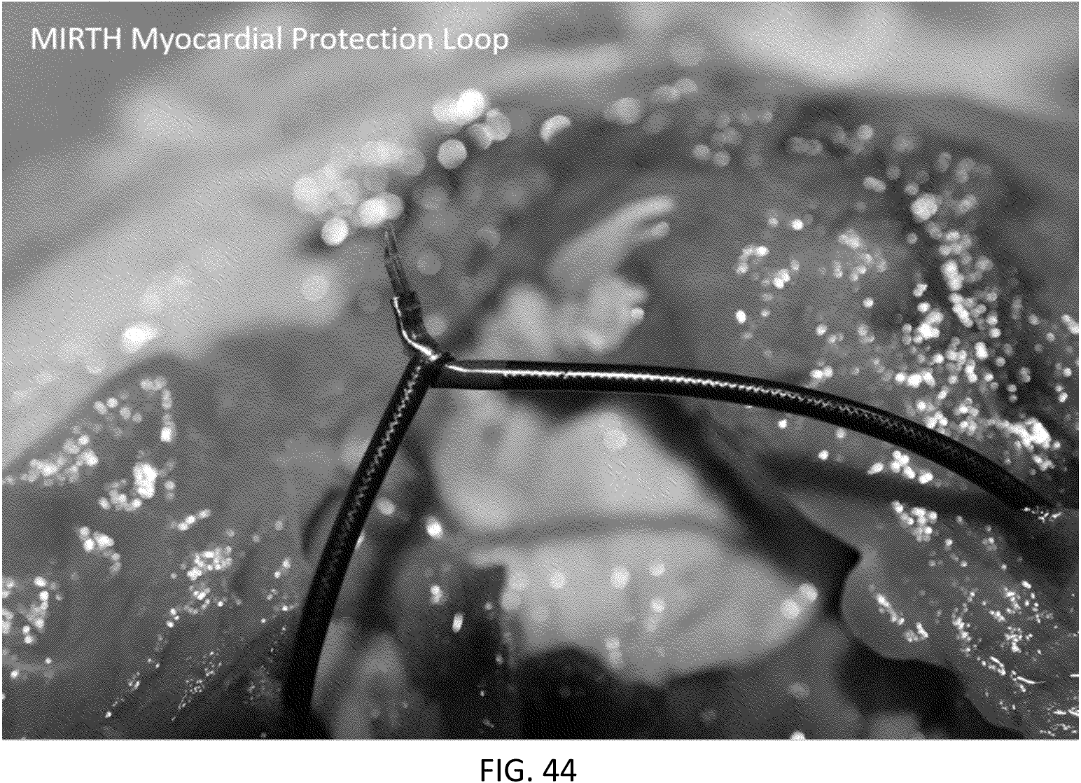

In some embodiments, the disclosure provides a method of reducing the dimensional size of a portion of a patient’s heart. An illustrative example of such a method is provided with reference to FIG. 25A to FIG. 44.

The method can include advancing a guidewire into a patient’s circulatory system and into the patient’s heart, advancing the guidewire through the myocardium to define a passageway around at least a portion of the heart between an outer surface of the heart and an inner surface of the heart.

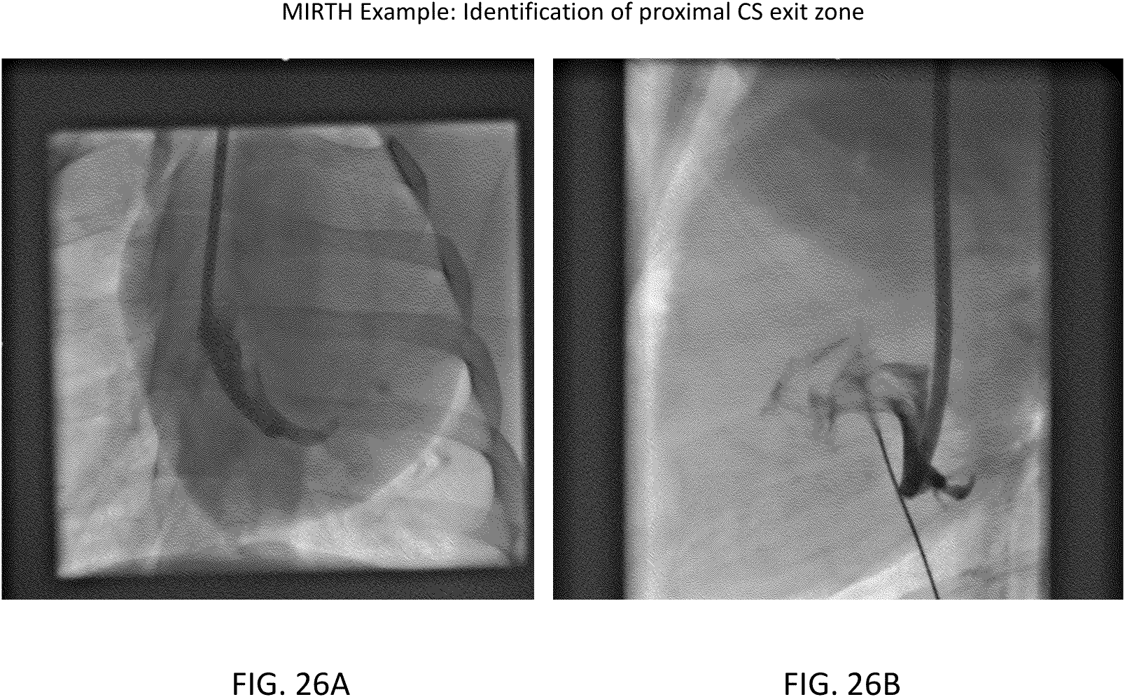

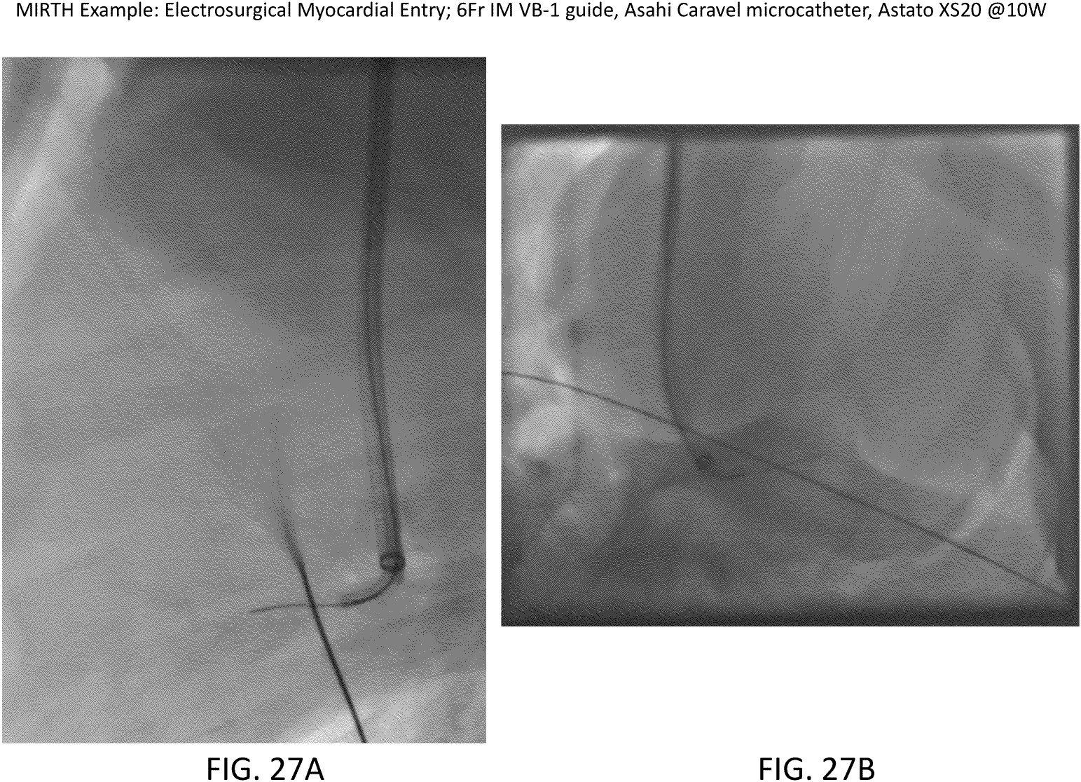

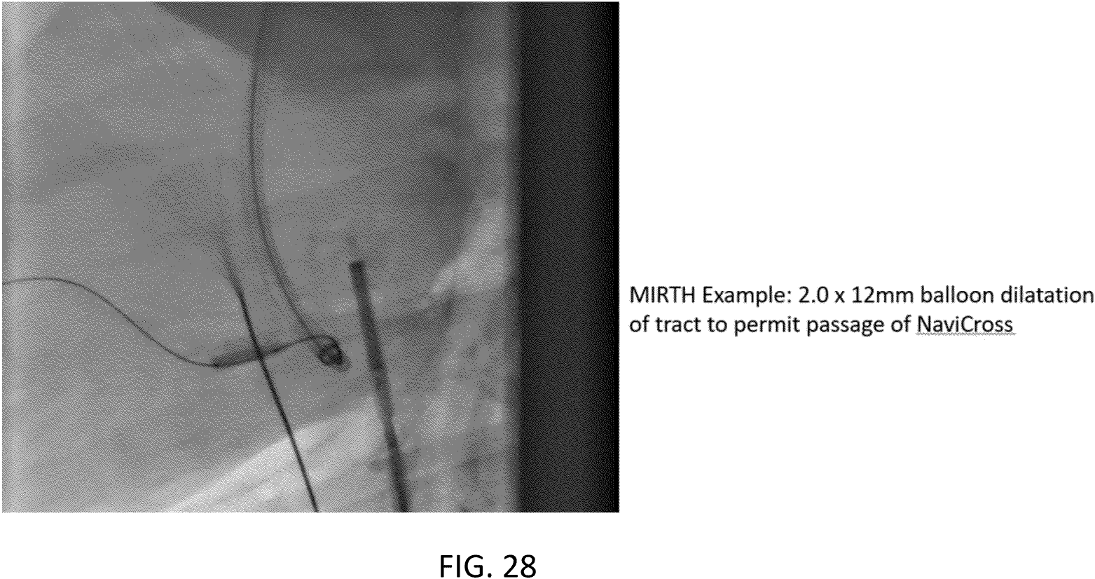

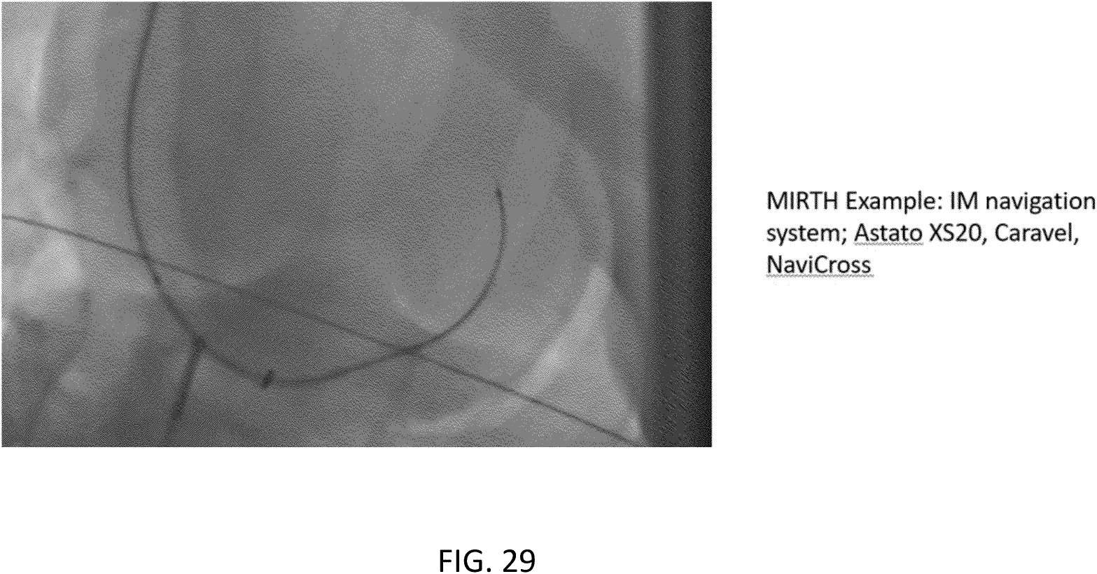

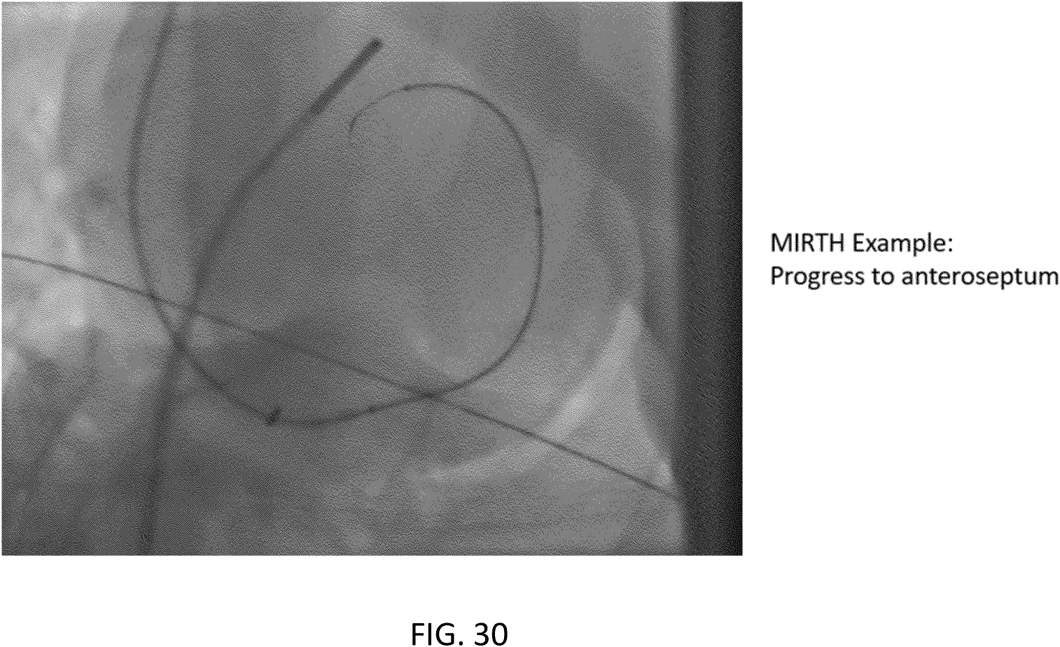

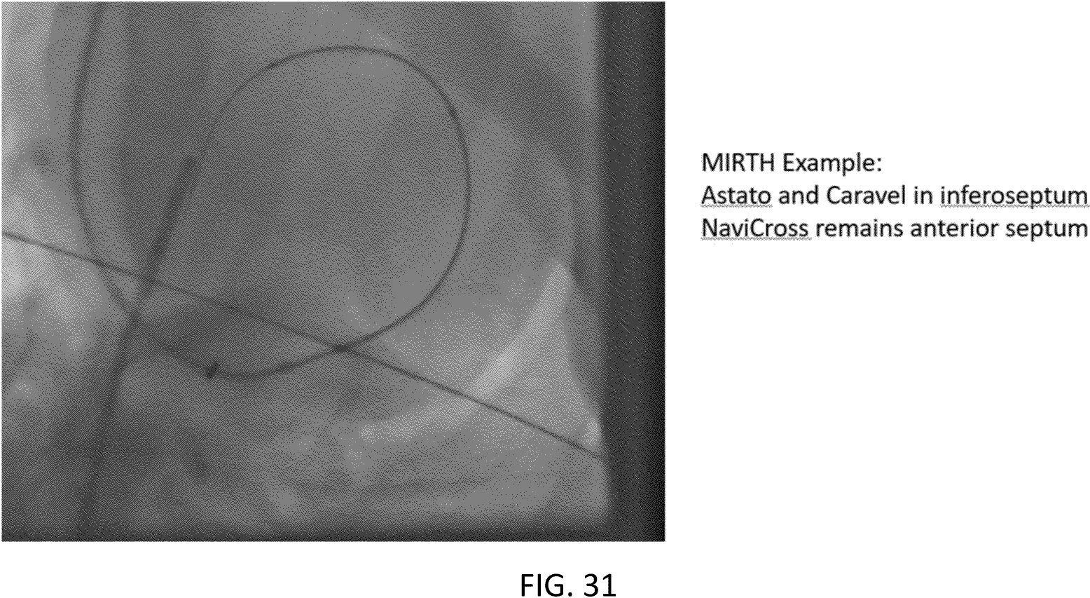

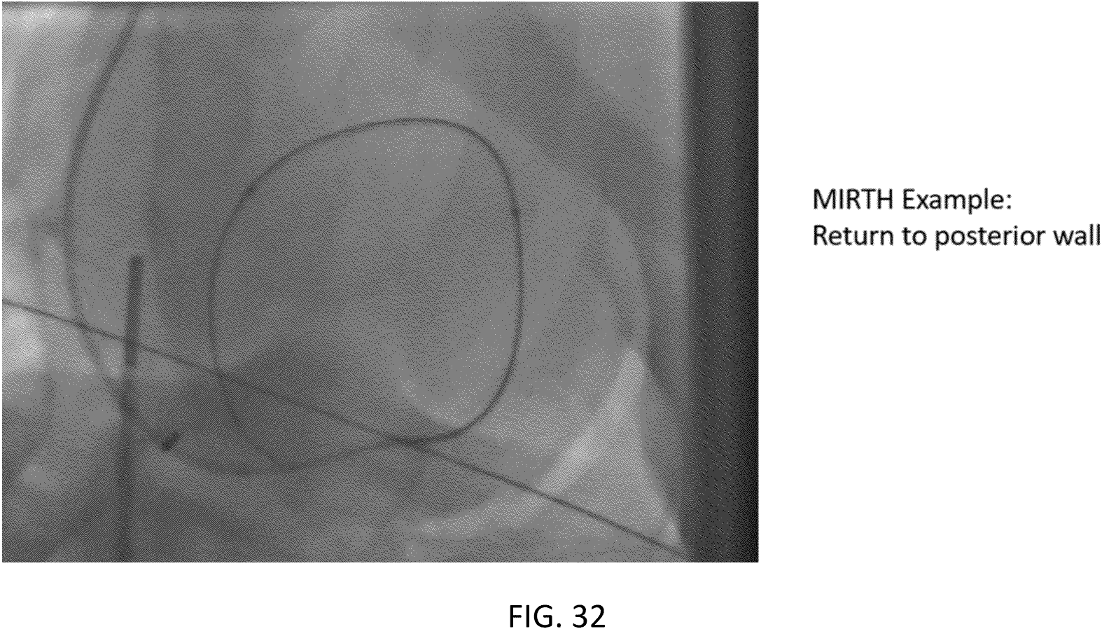

As illustrated in FIGS. 25A-B, the basal plane is identified on lateral projection, and the proximal CS exit zone is identified in FIGS. 26A-26B. FIGS. 27A-B illustrate electrosurgical myocardial entry, in this instance using a Asahi guidewire surrounded by a Caravel microcatheter, Astato XS20 @10 W. FIG. 28 illustrates an example of a 2.0 × 12 mm balloon dilatation of tract to permit passage of a Navicross® supporting catheter. FIG. 29 depicts the formation of the passageway by first advancing the electrified guidewire to ablate and/or bluntly dissect tissue. Dissection can be aided by using a pressurized fluid injected at the site to be dissected by the guidewire or microcatheter. FIG. 30 depicts the result of repeating this process as the passageway approaches the anteroseptum. FIG. 31 depicts the passageway defined as the Asahi and Caravel catheters progress into the inferoseptum as the NaviCross support catheter remains in the anterior septum. FIG. 32 depicts the guidewire passageway that is defined as the guidewire continues to define the passageway that returns to the posterior wall of the LV.

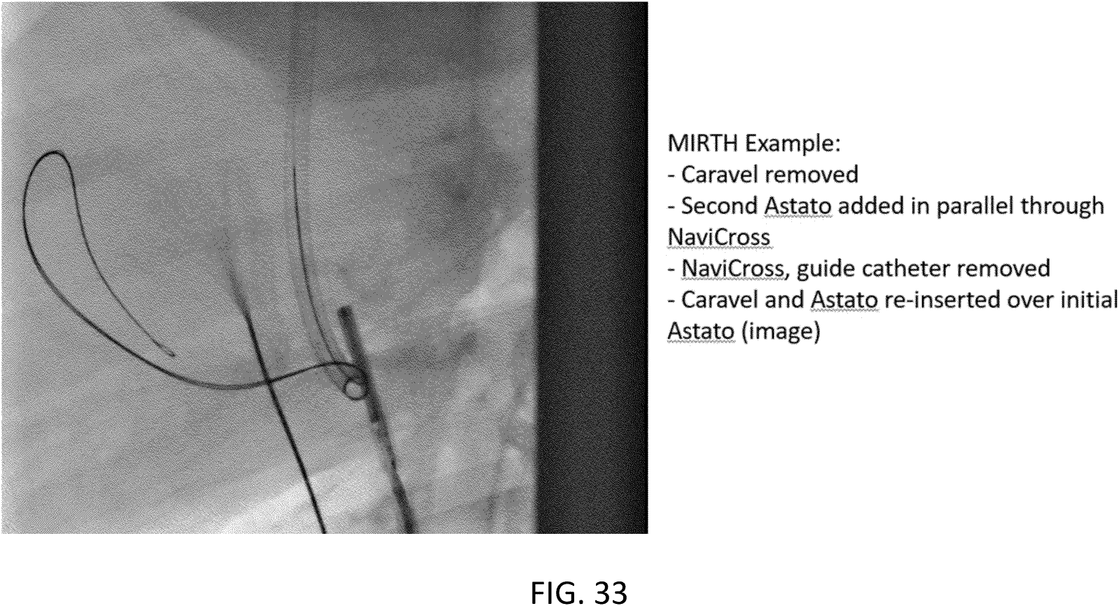

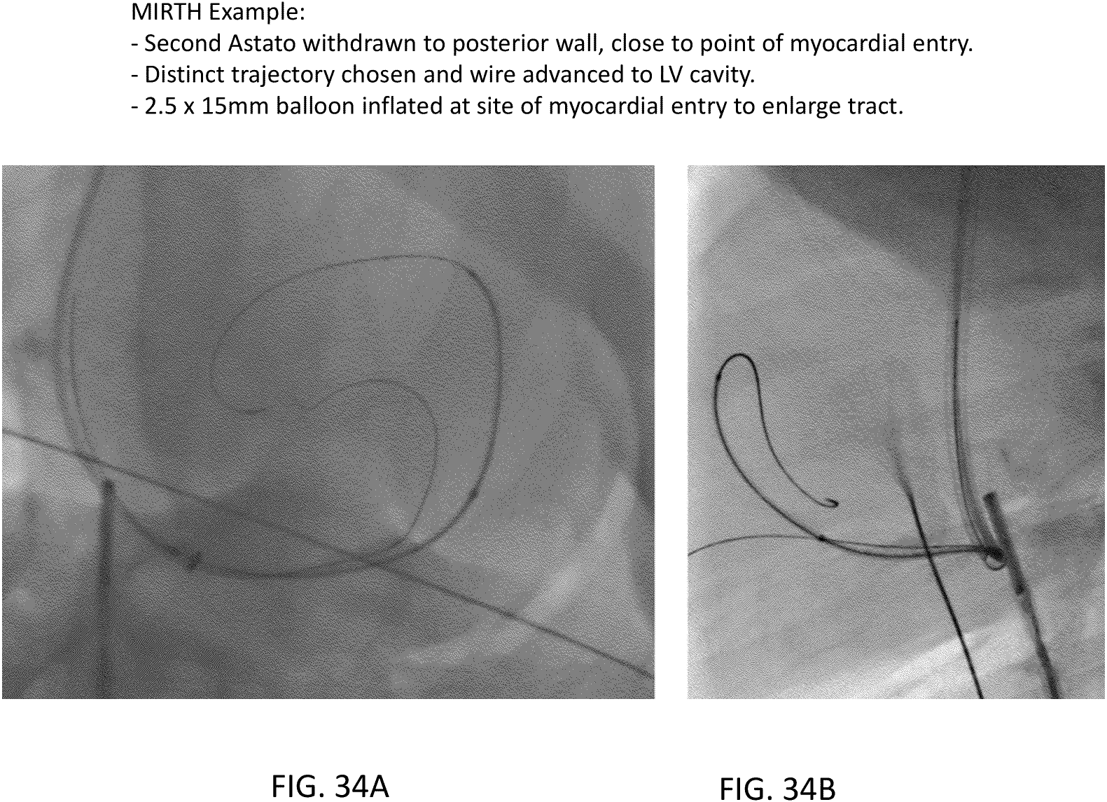

Next, as depicted in FIG. 33, the intermediate caravel catheter is removed to create an annular space between the NaviCross catheter and the guidewire, permitting a second guidewire to be introduced into the NaviCross. The NaviCross and guide catheter are removed, and the two support catheters are reinserted over the first guidewire, with the second guidewire alongside. As depicted in FIGS. 34A-B, and 35, the second guidewire is withdrawn along the passageway to the posterior wall, close to the point of myocardial entry of the first guidewire. A balloon catheter is introduced over this second guidewire to be inflated and enlarge the passageway near the entry point of the myocardium to enlarge the tract. Next, a snare catheter is introduced over the second guidewire, and deployed to capture the distal end of the first guidewire. The distal end of the first guidewire is then pulled and externalized to define a continuous passageway into the patient, around the LV, and back out of the patient.

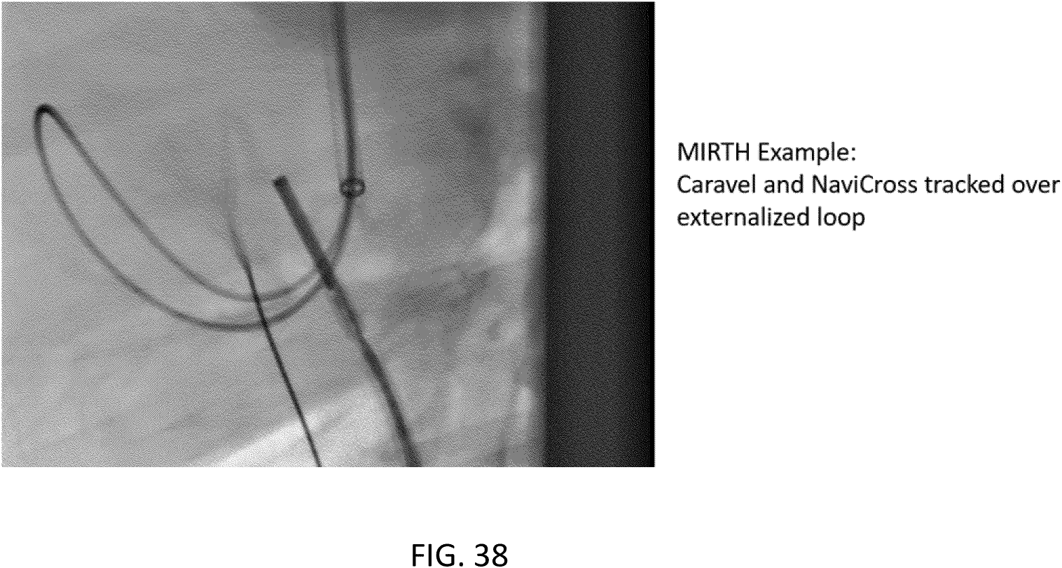

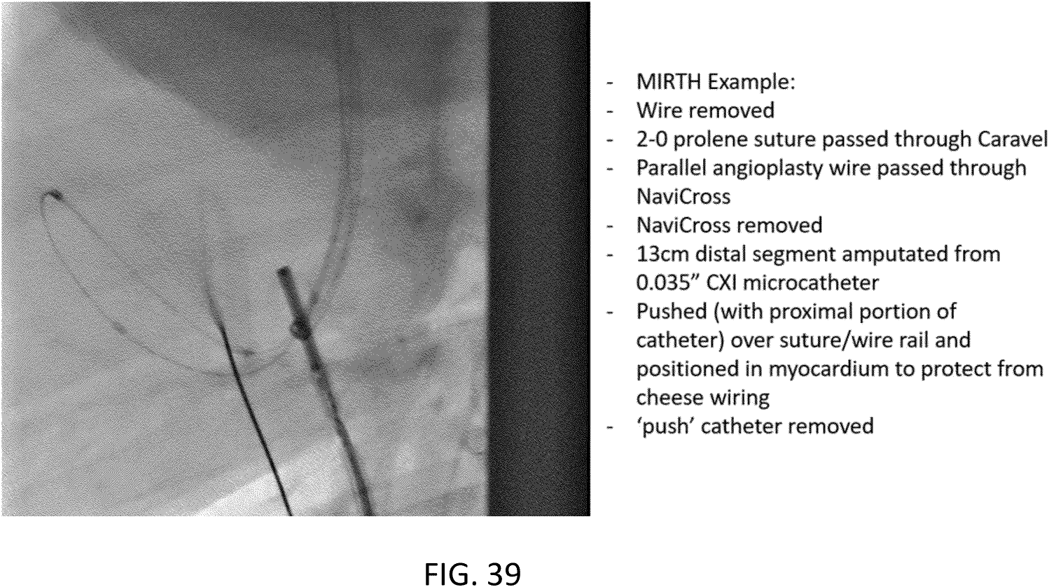

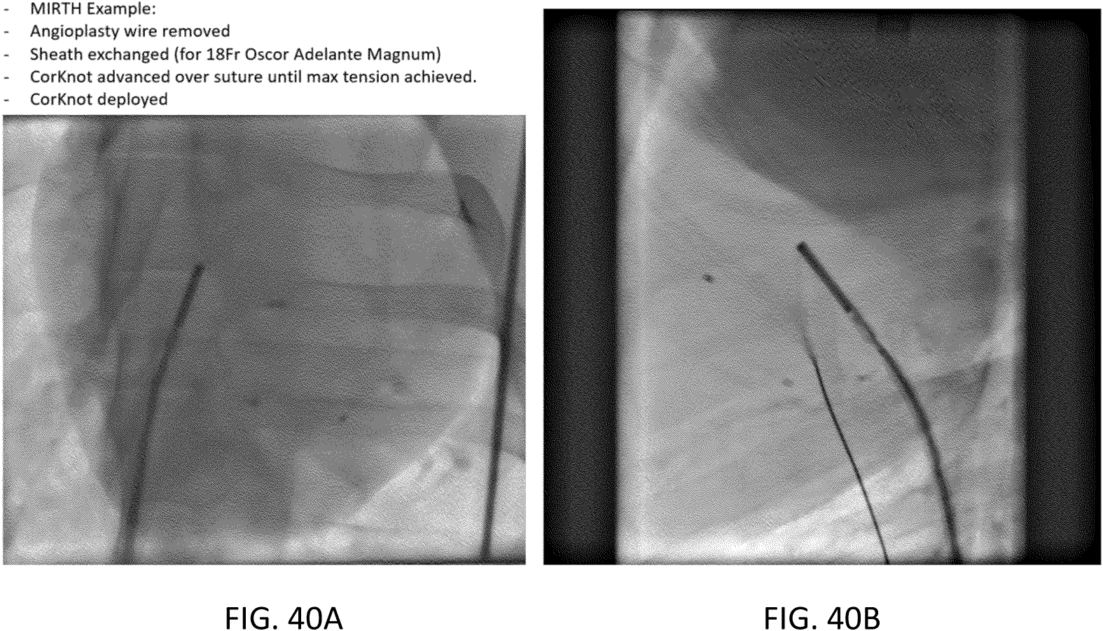

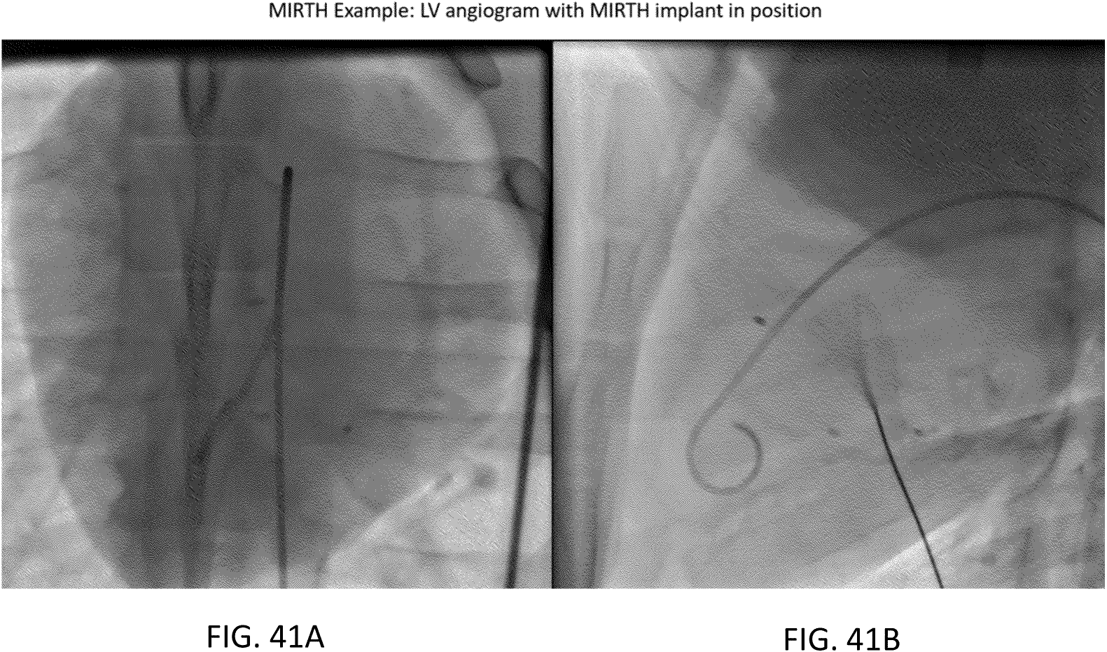

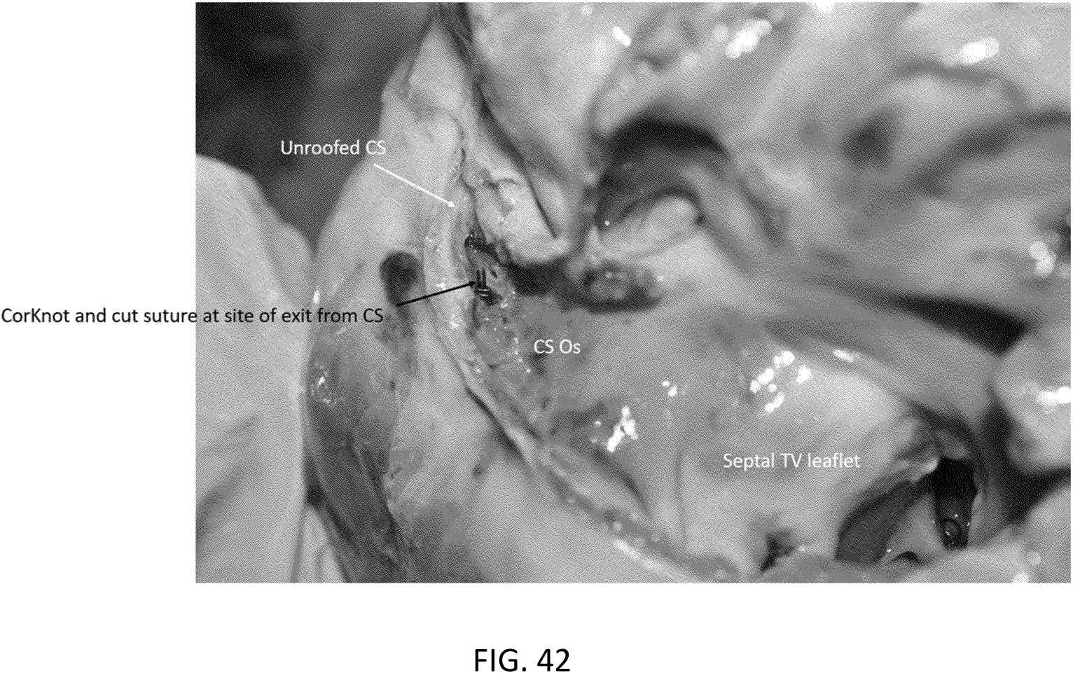

With the distal end of the first guidewire externalized, the intermediate (Caravel) and outer (NaviCross) catheter are advanced along the path of the first guidewire, and outside of the patient (FIG. 38). The distal end of the guidewire is attached to the tether of the implant to be installed, and the guidewire is withdrawn through the lumen of the intermediate catheter, pulling the tether with it. In this example, a parallel angioplasty wire is passed through the NaviCross, and the NaviCross is then removed (FIG. 39). A tubular member (distal catheter segment) is then pushed into the passageway around the ventricle, and the catheter used to push the tube to that location is then removed. This leaves a larger profile tubular member in place surrounding the LV. Then, as depicted in FIGS. 40A-40B, the angioplasty wire is removed, the guiding catheter is removed, and a knot is advanced over the suture until the desired tension is achieved. The excess material is cut off, and FIGS. 41A-B show the MIRTH implant in position. While this example shows a prototype implant, it is preferred to use the implant of FIGS. 1-4 herein as described above. FIGS. 42-44 show images of the heart removed after the procedure (on a porcine heart) showing the implant as installed with tissue cut away.

Generally, it will be recognized, in accordance with this disclosure, that any of the methods can include locking a lock over the suture, but then unlocking it, adjusting the tension in the tensioning element, and relocking the lock. In some implementations, the method can further include delivering a beneficial agent to a target location in the patient’s myocardium. In some implementations, delivering a beneficial agent can include performing a chemoablation procedure to debulk the myocardium. In some implementations, the beneficial agent can include one or more of (i) a pharmaceutical composition, (ii) light, and (iii) ultrasonic energy, for example.

In some implementations, the elongate passageway through the myocardium traverses a portion of the septum. If desired, the method can further include delivering a beneficial agent as described elsewhere herein to a target location in the patient’s septum. If desired, the delivering the beneficial agent can include performing a chemoablation procedure to debulk the septum.

In other implementations, the method can include defining an elongate passageway that traverses a path around a portion of at least one of the patient’s ventricles. If desired, the elongate passageway can traverses a path around a portion of both of the patient’s ventricles. If desired, the elongate passageway can encircle one of the patient’s ventricles at the basal level. In other implementations, the elongate passageway can encircle one of the patient’s ventricles at the mid-myocardial level. If desired, the elongate passageway can encircle the patient’s left ventricle.

In some implementations, the method can further include directing a second tensioning element through the patient’s myocardium and tensioning the second tensioning element to effectuate a further dimensional change to the patient’s heart. For example, multiple independent elongate passageways can be defined, and an implant can be installed along each elongate passageway.

Preferably, the procedures set out herein are percutaneous and the tensioning element can be introduced by way of the patient’s circulatory system. In some implementations, the procedure can includes advancing a guidewire percutaneously through a wall of a blood vessel in the heart and through the myocardium to define the elongate passageway. The procedure can include advancing a guidewire percutaneously through a wall of a blood vessel around the wall of the blood vessel to define the elongate passageway. The blood vessel can include the abdominal aorta, and the passageway can be defined through a healthy portion of the abdominal aorta located above an aneurysm, and further wherein the method can further include coupling the tensioning element to an implant disposed in the abdominal aorta to prevent the implant from migrating. The implant can be located in a manner so as to at least partially span, or fully span, a compromised region of the aorta, such as a region of the aorta that includes an aneurysm.

In some implementations, the method can further include directing a second tensioning element through the patient’s myocardium and tensioning the second tensioning element to effectuate a further dimensional change to the patient’s heart. For example, multiple independent elongate passageways can be defined, and an implant can be installed along each elongate passageway. This can effectuate a balance of forces to reduce the size of a patient’s heart and/or change its shape by applying forces across multiple directions through the myocardium. Likewise, any or all of such multiple implants can be provided with pacing or data collection capability by way of sensors to track and modify or treat a patient’s heart while it is still beating.

In some implementations, the lock can include an electrode array coupled to a signal generator configured to effectuate cardiac pacing, and the method can further include performing a cardiac pacing function using the electrode array and the signal generator. The pacing function can effectuate depolarization of the myocardium. In some implementations, the pacing function can include synchronously depolarizing the basal left ventricle. If desired, the pacing function can include effectuating a pacing function on the patient’s HIS bundle.

The disclosed methods can also include using electrode arrays embedded in the implant in order to sense particular locations in the tissue of the heart that are producing electrical signals, such as pacing signals, and then selectively ablating tissue so as to modify or eliminate those particular tissue locations from generating pacing signals, for example. If the pacing function of the native tissue is eliminated, the same and/or different electrodes can be used in order to provide pacing signals to the cardiac tissue as needed. The electrodes can also be selectively used to detect other electrical signals being generated by cardiac tissue and to depolarize tissue. In one implementation, an implant can be implanted so as to pace the HIS bundle, an electrode of an implant that is installed can be used to ablate tissue in the region of the AV node, and the same or other electrodes can then be used to provide pacing or other signals to the HIS bundle and effectuate HIS pacing.

The systems and methods of the present disclosure, as described above and shown in the drawings, among other things, provide for improved techniques for cardiac remodeling. It will be apparent to those skilled in the art that various modifications and variations can be made in the devices and methods of the present disclosure without departing from the spirit or scope of the disclosure. Thus, it is intended that the present disclosure include modifications and variations that are within the scope of the subject disclosure and equivalents.

1. An implant configured to traverse a passageway defined through tissue about a cardiac chamber of a heart, comprising:
a) an elongate flexible tether having two ends formed into a loop;
b) a lock body disposed over the two ends of the tether, the lock body being configurable of releasably engaging the elongate flexible tether; and
c) first and second tubular limbs extending outwardly from the lock over the elongate flexible tether along the loop toward each other.

2. The implant of claim 1, wherein the implant is compliant and can change in length in response to movements of the heart.

3. The implant of claim 1, wherein the first and second tubular limbs are of different diameters and have tapered distal ends.

4. The implant of claim 1, wherein a distal end of the first tubular limb slides within a distal end of the second tubular limb along the loop of the elongate flexible tether such that the first and second tubular limbs overlap.

5. The implant of claim 1, wherein at least one of the first and second tubular limbs includes a plurality of radiopaque markers along its length.

6. The implant of claim 1, wherein the plurality of radiopaque markers are disposed along the length of said at least one of the first and second tubular limbs in a predetermined pattern in order to facilitate measurement of the implant under visualization.

7. The implant of claim 1, wherein at least one of the first and second tubular limbs includes at least one pacing electrode to stimulate cardiac tissue.

8. The implant of claim 7, further comprising a controller coupled to the at least one pacing electrode to provide at least one of pacing, defibrillation, measurement and control.

9. The implant of claim 8, wherein the elongate flexible tether includes an antenna that conducts signals to and from the controller.

10. The implant of claim 1, further comprising a controller and a reservoir containing a beneficial agent, the controller being coupled to a dispenser that is coupled to the reservoir to dispense the beneficial agent.

11. The implant of claim 10, wherein the beneficial agent includes a medication.

12. The implant of claim 10, wherein the beneficial agent includes a gene therapy material.

13. The implant of claim 10, wherein the beneficial agent includes living cells to seed at least one location of the heart that is damaged.

14. The implant of claim 1, wherein at least one of the first and second tubular limbs includes at least one sensor to sense at least one biological parameter.

15. The implant of claim 14, wherein the at least one sensor includes at least one pressure sensor to sense blood pressure.

16. The implant of claim 14, wherein the at least one sensor includes at least one of: a chemical sensor, a distance sensor, a sensor having circuitry to detect electro physiological data, a movement sensor, and a location sensor.

17. The implant of claim 1, wherein the elongate flexible tether includes radiopaque material along its length.

18. The implant of claim 17, wherein the elongate flexible tether is a hollow braided suture, and the radiopaque material within the elongate flexible tether includes a radiopaque wire disposed within a length of heat shrunk polymeric tube that resides within a hollow core of the elongate inner tether.

19. The implant of claim 1, wherein an electrical circuit is completed in the process of locking the lock body into place.

20. An implant, comprising:
a) an elongate inner tether having a proximal end and a distal end, the proximal end of the elongate inner tether terminating in a loop; and
b) an outer tubular body surrounding the elongate inner tether along at least a portion of the length of the inner tether, wherein the outer tubular body is shorter in length than the elongate inner tether.

21-129. Please cancel without prejudice.
